(19) United States
(12) Patent Application Publication
(10) Pub. No.: US 2008/0287635 A1
(43) Pub. Date: Nov. 20, 2008

(54) Latex Adhesives Derived From Ionic Strength Induced Soy Protein Complexes

(76) Inventors: Xiuzhui Sun, Manhattan, KS (US); Li Xhu, Manhattan, KS (US); Donghai Wang, Manhattan, KS (US)

Correspondence Address: ERICKSON & KLEYPAS, L.L.C., 800 W. 47TH STREET, SUITE 401, KANSAS CITY, MO, 64112, US

(21) Appl. No.: 11/912,287

(22) PCT Filed: Apr. 24, 2006
(86) PCT No.: PCT/US06/15943

§ 371 Date: Jul. 22, 2008

Related U.S. Application Data
(60) Provisional application No. 60/674,176, filed on Apr. 22, 2005.

(51) Int. Cl. C07K 1/107

(52) U.S. Cl. 527/200

(57) ABSTRACT

Macro hydrophobic clusters and complexes of soybean globular proteins were observed using TEM (Transmission Electron Microscope). Upon unfolding, hydrophobic groups of the proteins became exposed toward the surface of the protein and actively interacted with other hydrophobic groups of other protein molecules, thereby forming hydrophobic bonding. The hydrophobic bonding resulted in hydrophobic protein clusters, the formation of which was affected by the degree of protein unfolding, protein structure, and hydrophobic components. Such hydrophobic clusters followed the global minimum free energy theory and formed spherical like structures with diameters ranging from 100 nm to 3000 nm. Such an understanding lends applicability to many uses in adhesives, molding composites, surfactants for oil-water systems, bio-based interior construction paints and paper coatings, fiber production, and metal powder molding applications.

### RELATED APPLICATIONS

This application claims the benefit of provisional application Ser. No. 60/674,176, filed on Apr. 22, 2005, the teachings and content of which are incorporated by reference herein.

### GOVERNMENTAL RIGHTS

The research was supported by a Grant No. DE-FC07-01-ID14217 from the Department of Energy. The government may have certain rights in this invention.

### BACKGROUND OF THE INVENTION

1. Field of the Invention

The present invention is concerned with protein polymers and bio-based adhesives. More particularly the present invention is concerned with protein polymers comprised of both hydrophobic and hydrophilic components, wherein the protein polymer is formed by unfolding the protein to a certain degree with either chemical, physical, or enzymatic methods. Upon unfolding, the hydrophobic components of the protein are revealed, resulting in a sticky and highly interactive surface area on the protein, and forming surface active and interactive protein polymers (SAIPP), which can then be used in liquid, semi-liquid, or powder form, alone or in combination with a number of different compounds, such as cellulose-based materials, latex-based adhesives, or thermoplastic resins. The present invention is also concerned with using such SAIPP in combination with other hydrophobic polymers or compounds. Still more particularly, the SAIPP can be used in a variety of applications including 1) adhesives for wood veneer, foundry applications, children's glue, adhesive labels, packaging sealant, and the like; 2) hot melt adhesives wherein the SAIPP can be used alone or be prepared into powder form and blended with hydrophobic polymers to form hot melt adhesives that are highly water resistant; 3) molding composites wherein the SAIPP can be used in powder form and blended with other polymers and fibers; 3) as a macromolecular surfactant for an oil-water system; 4) bio-based interior construction paint and paper coating, wherein the SAIPP can be used as homo- or co-polymers in liquid form; 5) fiber production wherein the SAIPP can be used alone or in combination with other polymers; 6) metal powder molding applications wherein the SAIPP is used as an aqueous de-bonding polymer; and 7) to form latex-like adhesives with strong wet-tack properties for a variety of adhesive applications.

2. Description of the Prior Art

Anfinsen published a paper in Science in 1973 proposing the “thermodynamic hypothesis” that governed the protein folding. (Anfinsen, Christian B. Science, 1973, 181 (4096), 223). This theory suggested that protein folding followed the principle of global minimum that is so called minimum free energy theory. Van Holde disagreed with this concept and believed that protein folding was affected by amino acid composition and their sequences as well as microenvironment factors that were predominated by biologic functions (Van Holde, K. E. In Food Proteins Eds., Whitaker, J. R. and Tannenbaum, S. R. AVI Publishing Company, Inc. Westport, Conn., 1977, Chapter 1, Page 1). More recently, it has been proposed that the tryptophan residue is the key to forming the network of hydrophobic clusters in hen lysozyme protein. The hydrophobic cluster causes the protein folding, and such hydrophobic clusters in denatured proteins can be detected by NMR techniques (Baldwin, Robert L., Science, 2002, 295, 1657). These hydrophobic clusters are not only formed in water but also in the presence of denaturing agent, such as urea. Under this theory, at least one tryptophan residue is needed for such a cluster formation.

With respect to the bio-based adhesive and coating applications of the present invention, each year, billions of pounds of adhesives and coatings including latex-based adhesives, foundry adhesives, wood adhesives, all-purpose glues, labeling adhesives, hot melt adhesives, paints, paper box packaging adhesives, and envelope adhesives are used. Most non bio-based adhesives and coatings contain more or less hazardous chemicals, such as vinyl acetate or acetaldehyde and other chemicals, that may cause skin and eye irritation. Additionally, the vapor produced from adhesives during processing and use may cause respiratory tract irritation. Long term exposure to these adhesives and coatings containing hazardous chemicals may even be carcinogenic. In a study where lab animals were exposed to 600 ppm of latex-based adhesives over their lifetime, it was found that vinyl acetate vapor caused tumors in the respiratory tract, and exposure to only 200 ppm caused respiratory tract irritation. At this time, approximately 200 million bottles of all-purpose glue, such as Elmer's®, are used annually in the United States, particularly in grade schools, day-cares, homes, and offices. Most of the all-purpose glues are prepared using latex-based formulas. In fact, although Elmer's® Glue is defined as a non-toxic substance in the Federal Hazardous Substances Act, its Material Safety Data Sheet indicates that prolonged skin exposure or inhalation can cause irritation. Prior to the present invention, no bio-based adhesives existed that performed comparably to latex adhesives.

A large market for latex-based adhesives is in the labeling of all kinds of containers, as well as the production and assembly of packaging containers, and paper boxes. Label adhesives with wet tack properties are able to adhere a label to a bottle immediately, and are strong enough to hold the label in place to survive a fast processing line. Industries usually remove the labels before recycling. Often times, the labels must be removed by burning the bottles, which consumes substantial energy and releases unpleasant vapors into the environment.

Sand adhesives are used in the metal alloy, iron, and steel casting foundry industries and, as a result of processing, generate huge amounts of air pollution due to hazardous substance emission. The used sand must often be reconditioned for recycling, and the core must be removed after casting.

Accordingly, what is needed in the art are methods of forming improved adhesives. What is further needed are methods of forming surface active and interactive protein polymers for use in a variety of applications. What is still further needed is an adhesive derived from a renewable source, which has adhesion and water resistance properties comparable to conventional latex adhesives, and methods of making the same. What is even further needed is a protein-based adhesive having similar water resistance and adhesion properties as a conventional latex adhesive, and methods of making the same. What is further needed are adhesives utilizing proteins derived from biological sources, such as vegetable, grain, or animal proteins that have been modified to increase their adhesive strength and water resistance, and methods of making the same. What is still further needed in the foundry industry are adhesives that decompose at temperatures above 250° C., while remaining odorless. What is even further needed in this respect are adhesives that need little to no reconditioning when recycling used sand, and which require no labor to remove the core because all of the sand is removable by air or a mechanical pumping system. What is still further needed are SAIPP useful in coatings, hot melt adhesives, paints, paper coatings, textile fabrication, wood veneers, and bio-based macromolecular surfactants.

### SUMMARY OF THE INVENTION

The present invention overcomes the problems inherent in the prior art and provides a distinct advance in the state of the art. In particular, the invention provides for protein-based adhesives and polymers and methods of making and using the same. The protein-based adhesives and polymers of the present invention are comprised generally of novel SAIPP. The SAIPP have a variety of potential applications, and the various physical or chemical properties of the polymers can be easily manipulated in accordance with a desired use. For example, the SAIPP can be designed to produce novel adhesives that are “latex like,” meaning that they have wet tack properties and remain sticky to adherent surfaces with sufficient gluing strength when wet. These protein-based wet-tack adhesives provide all of the benefits of a latex adhesive with none of the drawbacks. Advantageously, adhesives in accordance with the present invention cure within a few minutes at both room and elevated temperatures, and are also microwave curable. Additionally, adhesives in accordance with the present invention retain their stickiness and adhesive properties upon subsequent re-wetting. There are large numbers of potential materials to which the latex-like protein adhesives of the present invention will adhere, including but not limited to, paper, cloth, wood, cellulosic-based materials, fiber cardboards, glass, sands, and plastics.

The SAIPP of the present invention can be used alone as an adhesive, or can be combined with polymers, fibers, cellulose or other materials in accordance with a variety of further applications. For example, the SAIPP of the present invention can be prepared in powder form and blended with hydrophobic polymers to form hot melt adhesives that are highly water resistant, or blended with other polymers and fibers for molding composites, fiber production and artificial wood products. In another application, the SAIPP of the present invention can be used as a macromolecular surfactant for oil-water systems. The SAIPP of the present invention can also be used in liquid form as homo-polymers or co-polymers for bio-based interior construction paint and paper coating. The SAIPP of the present invention also have potential in metal powder molding applications as they can function as aqueous de-bonding polymers. Accordingly, the SAIPP of the present invention have a great number of potential applications.

The ability to manipulate SAIPP in accordance with the present invention is made possible through an understanding of proteins, and their interaction under certain conditions and in the presence of other modifying reagents, chemicals, compounds, and the like. Proteins are complex macromolecules that contain a number of chemically linked amino acid monomers, which together form polypeptide chains, constituting the primary structure. The a helix and β sheet patterns of the polypeptide chains form the secondary structure. A number of side chains are connected to these amino acid monomers, and interact with each other mainly through hydrogen and disulfide bonds forming tertiary, or quaternary structures. Amino acid residues can be categorized as either hydrophobic or hydrophilic. Hydrophobic residues, such as Ala, Phe, Met, Cys, Val, Leu, Ile, etc., form hydrophobic clusters when they contact each other, causing protein folding. In an aqueous system, the hydrophobic clusters pack tightly together and bury deep inside the protein away from the water. Hydrophilic residues such as Lys, Asp, Glu, Arg, Pro, Gln, Asn, His, etc., are important amino acids that remain on the outside of the protein, thereby enabling the protein to dissolve in aqueous systems. Once a protein is denatured, its water solubility often becomes lower due to the exposure of some of the hydrophobic components. Final conformation of a native protein can be determined by its amino acid composition, sequence and biological function. However, as denatured proteins often lose their biological functions, re-assembly conformation of a denatured protein is highly affected by amino acid composition, sequence, and surrounding environment, such as solvent, pH, temperature, ion concentration, chemical composition and the presence or absence of enzymes.

Proteins can be modified or denatured using physical, chemical, or enzymatic methods resulting in structural or conformational changes from the native protein structure without alteration of the amino acid sequence. Protein modification may increase a protein's tendency to unfold, thereby revealing the hydrophobic core and, consequently, increasing the bonding strength. Protein modification may also move the hydrophobic amino acids outwards to increase water resistance. Proteins containing hydrophobic and hydrophillic amino acids were found to have positive effects on gluing strength and water resistance in the adhesives and polymers of the present invention. The desired hydrophillic/hydrophobic content and subsequent modification or unfolding of SAIPP in accordance with the present invention will depend upon the desired end-use of the protein, but will be determinable by those of skill in the art due to the understanding of protein interaction provided by the present application.

The present application demonstrates that a hydrophobic cluster can be formed by the interaction of a selected group of hydrophobic amino acids, such as Ala, Phe, Leu, Val, Ile, Tyr, Trp, and Met, regardless of the presence of tryptophan. The present invention further demonstrates that the formation of “macro hydrophobic clusters” is caused by hydrophobic globular polypeptides interactions that can be induced by unfolding agents such as an ionic compound, salt, reducing agent, or detergent in an aqueous system. The term “macro hydrophobic cluster” is defined herein as the clusters formed by inter protein-protein interactions. The driving force for protein folding is provided by water so that the hydrophobic clusters exclude water and tightly pack together and bury themselves inside of the protein. Therefore, the surface of most proteins in water is hydrophilic and it is impossible for such a macro protein with a hydrophilic surface to interact with other proteins forming a macro hydrophobic cluster in water. However, once the protein is unfolded or denatured and turned inside out, the surface of the protein becomes more hydrophobic, thereby promoting inter protein-protein interaction and forming macro hydrophobic clusters.

For the present invention, soybean storage proteins were selected for testing because they are globular proteins and easily separated, and their amino acids and sequences have been thoroughly studied and well known. Soybean storage proteins have been recently considered as potential alternative polymers for industrial applications to reduce dependence on petroleum resources and decrease environment pollution. Soybean is an oilseed having a storage protein content of about 40%. Like many globular proteins, soybean proteins are made of the 20 various amino acids forming primary, secondary, tertiary, and quaternary structures. Soybean proteins consist of many polypeptide chains. Glycinin (˜30%) and conglycinin (˜30%) proteins are major polypeptide chains of soybean proteins. Glycinin protein, with a molecular weight of 300-380 kDa, has six major sub-polypeptides and each of them contains a pair of acidic and basic subunits that are alternatively linked by disulfide bonds forming a hexamer. Conglycinin protein, with a molecular weight (MW) of 150-200 kDa, contains four sub-polypeptides including α, α′, β, and γ. The sub-polypeptides of conglycinin are held together primarily by hydrophobic forces and hydrogen bonding. The structure, surface properties, and conformation of a globular protein are sensitively influenced by its environmental conditions, such as pH, ionic strength, and temperature.

Macro hydrophobic clusters and complexes of soybean globular proteins were observed using TEM (Transmission Electron Microscope). Upon unfolding, hydrophobic groups of proteins were exposed toward the surface of the protein. Those hydrophobic groups actively interacted with other hydrophobic groups of another protein molecules, thereby forming hydrophobic bonding. The hydrophobic bonding resulted in hydrophobic protein clusters. The hydrophobic cluster formation was affected by the degree of protein unfolding, protein structure, and hydrophobic components. Such hydrophobic clusters follow global minimum free energy theory and in one experiment, formed spherical-like structures with diameters ranging from 100 nm to 3000 nm. In this experiment, the protein unfolding was obtained by using sodium bisulfite reducing agent. At low concentrations of the reducing agent (i.e., at ionic strengths less than or equal to 0.05766), the hydrophobic clusters can be prevented from forming complex structures by manipulating the pH of the protein solution. However, at high concentrations (i.e., at ionic strengths equal to or higher than 0.1153), some of the hydrophobic clusters cannot be destroyed, as they formed large clusters up to 3000 nm in diameter. Because of these hydrophobic clusters, the aqueous protein liquids had shear-thinning and temperature-dependent flow behavior. Upon curing, phase separation was observed using LSM (laser scanning microscope) techniques. Small amounts of hydrophilic components were squeezed out, thereby forming lines due to hydrophobic protein aggregation.

According to these discoveries it was determined that SAIPP could be formed for uses as adhesives and polymers in various applications. A preferred method of protein modification occurs in solution. More preferably, protein unfolding occurs in an aqueous solution. Depending on the ionic strength of the solution, a protein in solution forms either a trimer or hexamer structure. For example, conglycinin protein (7S), from soy, becomes a trimer structure at an ionic strength of I>0.5 and a hexamer structure at an ionic strength of I<0.2, in solution with pH ranging from about 4.8 to about 11 (Information from Protein Data Bank). In some cases, a water channel can be formed at the center of the protein trimer structure, whereby the surface of the protein becomes more hydrophobic (Adachi et al., 2001). Due to ionic attraction forces, ions in solution will attract positively- or negatively-charged functional groups of the protein outward, causing the compact protein to swell and unfold. Some of those functional groups, such as carboxyl groups, are sticky. It was discovered that the swollen and unfolded proteins would become entangled and interact with each other upon applying a certain amount of mechanical force or by condensation methods, or by unfolding the protein in high-solids concentration (i.e. 40% solids content), thus forming a continuous complex. It was also determined that the continuous complex with its sticky functional groups had wet-tack adhesion properties. Variable parameters affecting adhesion performance, such as the degree of protein swelling and unfolding, protein surface structure, functional groups and their concentration on the protein surface, can be controlled by changing the ion types and ion concentration in the solution, protein structure, protein molecular size, distance between protein molecules, and mechanical force applied.

While studying soy protein isolates, it was found that the basic components of glycinin protein from soy protein have a much higher wet strength than the acidic components because the basic components contain more hydrophobic amino acids. It was discovered that native corn zein protein, containing large amounts of hydrophobic amino acids, also has strong gluing strength and high water resistance. It was also determined that the neutral surface charge of a protein (obtained by adjusting pH to isoelectric point, pI) improved the wet strength of the protein-based adhesive. For example, an adhesive prepared from a native soy protein at about 14% solid content, a pH of about 4.6 (isoelectric point), and cured at about 180° C., had a dry adhesive strength of about 6.7 MPa and a wet strength of about 3.1 MPa. It was also determined that a protein peptide containing a hydrophobic core flanked with positively charged lysine residues had strong adhesion properties.

In a general method in accordance with the present invention, SAIPPs are prepared using a protein or protein polymer containing both hydrophobic and hydrophilic components. The proteins can be either a mixture of several polypeptides or a homogenous system with one polypeptide. The polypeptides can be defined as hydrophobic polypeptides if they contain at least about 40% or more hydrophobic components (i.e. aliphatic, aromatic or sulfur containing groups), based on total amino acid content; and as hydrophilic polypeptides if they contain at least about 60% or more hydrophilic components (i.e., bases, acids, amides, or alcohols groups). Protein composition and polypeptide structure can be selected from naturally occurring proteins, such as plant proteins (i.e., soybean, corn, wheat, other cereals and oilseeds) and animal proteins. The selection of the protein is not affected by its source, but rather by the amino acid content or composition therein. However, for ease of working with characterized proteins, preferred proteins are plant proteins. More preferably the protein is a vegetable protein, and most preferably, the protein is selected from the group consisting of soy and canola proteins. The protein compositions and the structure of the polypeptide chains can also be designed by genetically engineering selected crops. Specific methods of protein modification or denaturing will be apparent to those in the art and specific guidance is provided in the examples below.

In one embodiment of the present invention, there is provided a method of making SAIPPs by unfolding a selected protein to a particular degree depending on the protein and particular application. The protein can be any protein containing both hydrophilic and hydrophobic components, in any form including but not limited to all meals containing protein from oil production, starch production, ethanol production, protein flour, powder, protein meal or protein isolates. Once selected, the protein can be placed in water, such that the solution has a resulting solid content of preferably about 1% to 40% solids. For plywood or sprayable adhesives, 5-15% solid content is preferred. For roll coating, curtain, wood veneer, sheet, or foundry applications, 15-30% solid content is preferred. 20-40% solids content is preferred for labeling or coating applications. 90-100% solids content is preferred for powder forms. The water can be from any source, but preferably the water is distilled water or tap water. The pH can be adjusted according to the desired application to a value that promotes unfolding. This desired pH level will also depend on protein structure and unfolding agent. An unfolding agent, such as urea, detergents, reducing agents or salts is added and the solution is stirred. Preferably, the protein unfolding agent is selected from sodium salts. The amount of unfolding can be varied according to a particular application. High unfolding results in a more cohesive polymer, whereas less unfolding results in a more adhesive polymer. Preferably quarternary and tertiary structures are unfolded with the attendant destruction of hydrogen and disulphide bonds while maintaining the secondary structure such as the alpha helix and beta sheets. The unfolded proteins then interact with each other, or can be reacted with other hydrophobic polymers or compounds to form a complex polymer. Upon unfolding, some of the hydrophobic and charged groups, including either aliphatic, aromatic, or sulfur containing groups, become redistributed and the surface of the protein becomes sticky, and highly reactive and/or interactive. The pH is adjusted again according to the desired application. By adjusting the pH, the SAIPP's hydrophobic-hydrophilic functions, and thus its level of stickiness, can be altered in accordance with a specific application. In one aspect, the pH is adjusted to be near the isoelectric point of the protein. In another aspect, the pH is adjusted to be at exactly the isoelectric point of the protein. In yet another aspect, the pH is adjusted to be neutral. For water resistant adhesives or coatings, the pH is generally adjusted to be near or at the isoelectric point of the protein. Preferably this is within +/−0.2 of the pH of the isoelectric point. For gelatins or surfactant applications, the pH is unrelated to the isoelectric point.

Once formed, the resultant SAIPP can be used as a liquid, semi-liquid, or in powder form depending on a desired application, and in particular with the examples contained herein. For example, the water content can be reduced to anywhere between 10% to 99%, through evaporation or centrifugation, with a resulting solid content of about 1% to about 90%, depending on the application. In particular, protein solutions with lower water content can be used as powders and blended with other materials. Protein solutions with higher water content can be used in aqueous applications or as adhesives per se. Variations in accordance with the present invention can be readily determined by those of skill in the art without undue experimentation.

In a preferred embodiment of the invention, the SAIPP can be prepared in powder form and subsequently blended with thermoplastic polymers/resins. Preferably, the thermoplastic polymer/resin will contain at least one functional group selected from the group consisting of CH₃, OH, COOH, NH₂, SH, etc., per chain length. Preferably, the polymers that are added such as polylactic acid are either aromatic or aliphatic polymers. The blends can be prepared at the melting temperature of the thermoplastic polymer, preferably from about room temperature to about 230° C. For example, blends with Elmers Glue are done at room temperature, while blends with polylactic acid are between 170-185° C. For polyvinyl acetate blends, blending can be done at 140-180° C. The resultant blend can be used as a hot glue gun adhesive or extruded into thin noodles or sheets for other hot melt adhesive applications. The blend can also be cured by cold press at about room temperature. In another aspect, the adhesive can also be used as a resin, and blended with fibers in an extruder for molding composite products. Preferably, coupling agents, selected from the group consisting of methylene diisocyanate (MDI), maleic anhydride, or methyl acrylate (MA), with reactive functional groups including CH₃, OH, COOH, NH₂, SH, etc., are used to improve the properties of the blends between SAIPP and other polymers. In still a more preferred embodiment, polylactic acid (PLA) can be blended with SAIPP in accordance with the present invention at a ratio of SAIPP to PLA of about 30:70 with SAIPP being from 5 to about 50% of the composition. Preferably, the blend includes a coupling reagent such as MDI, or a coupling reagent containing amine groups. These coupling reagents may be present in the composition from about 0.1 to about 5%. Additionally, the SAIPP are preferably uniformly dispersed in the PLA matrix such that the PLA's flowability is significantly improved and the resultant blend is viscous and sticky. In another preferred aspect of the present invention, poly vinyl acetate-based resins and polymers are used in accordance with this method.

In still another embodiment of the present invention, the SAIPP are prepared in aqueous form and blended with either hydrophobic or hydrophilic polymers or resins in liquid form for adhesives and/or paint applications. The polymers or resins can be either aqueous or nonaqueous. In particular, SAIPP in liquid form can be blended with children's glue (e.g., Elmer's® Glue, or the like) to reduce the use of latex glues for children.

In still another embodiment, SAIPP in accordance with the invention are prepared in aqueous form and blended with an epoxidized plant oil, such as epoxidized soybean oil (ESO). The ring of the ESO can be opened using a catalyst, such as BF₃, and the opened ESO can be blended with the SAIPP. The NH₂ groups from the SAIPP act as curing agents of the ESO. Coupling reagents or curing agents for ESO can also be added. In a preferred aspect, the SAIPP can also be blended with ESO directly or with ratios of ESO with ring-opening ESO for self-healing paint materials.

In another embodiment of the present invention, there are provided latex-like protein adhesives. One of the preferred proteins for use in accordance with the present invention, and specifically with the latex-like protein adhesives, is soy protein and derivatives thereof. In order to study soy protein structures and adhesive properties, conglycinin subprotein (called 7S) was separated from soy protein isolates. A number of known methods for 7S purification were attempted before arriving at the novel method of the present invention. A method for 7S purification, described by Thanh and Shibasaki (1976) (“TS method”) was employed, with some modification. According to the TS method, 0.3 mole Tris-HCl was used as a buffer containing 0.01 mole of 2-mercapto-ethanol (2ME) in a 0.02 soy protein solution. The mixture was stirred for about one hour at room temperature and then was centrifuged at 10,000 rpm for 20 minutes. To reduce 2ME concentration (due to its unpleasant odor), the TS method was modified by adding 0.1 N sodium bisulfite (NaHSO₃) to balance the ionic strength. However, the resulting precipitates were normal proteins with no tack and/or viscous properties and deemed unsuitable for the present invention. Another method for 7S purification, described by Fukushima (1968) was similar to the TS method for 7S purification, but omitted Tris-HCl. It produced the same results. Another commonly used procedure to extract 7S and 11S out of the soy protein isolates was developed by Nagano (1992). In this procedure, 0.98 g NaHSO₃ per liter soy protein solution was used at a desired pH, stored at 4° C. for 12 hours, and then centrifuged at 6500-9000 g for 30 minutes. This procedure was also used for 75 and 11S extraction from soy flour by Sun et al (1999). The precipitated 7S or 11S had no “latex glue” behavior.

The present invention improves upon known methods in the art, and uses 2ME at 0.01-0.02 mMole/50 g soy flour and 1.01 g NaHSO₃ per liter of slurry volume for 7S extraction, hereinafter known as the “Sun method.” Upon examining the effect of stirring time using the Sun method on 7S purity, it was found that the precipitation of 7S did not produce glue properties until one of the samples was inadvertently left in a refrigerator for over 24 hours. After undergoing centrifugation, the precipitated protein unexpectedly became a “latex-like” adhesive. In a more preferred embodiment, the procedure described in Example 3 was followed, and the same results were obtained. No prior art for this method or its results exists.

Ionic strength, centrifuge intensity, and protein composition are the most important factors for producing the latex-like soy adhesives. In another preferred aspect, 2ME was removed, and the sodium bisulfite concentration was increased up to 6 g NaHSO₃ per liter protein solution (usually at about 5% solid concentration) and the centrifuge intensity was adjusted (see, Example 6) to obtain results similar to those described in Example 3. Because sodium bisulfite is a commonly used food preservation agent, the present method has further potential applications for edible composites. To reduce storage time, a short procedure was developed (Example 12), resulting in yet another aspect of the present invention. Sodium hydroxide was used to unfold the protein to a certain level and then the NaHSO₃ interacted with protein functional groups and obtained the similar results without needing the 24 hour storage time. Such a procedure is novel and no prior art exists.

In a preferred embodiment of the present invention, the SAIPP in liquid form as a latex-like adhesive composition comprises a protein and an unfolding agent (such as an ionic compound) is provided, wherein the pH of the composition can be adjusted to be near a desirable value depending on the application and protein structure, unfolding agent used, other proteins present in the system, or the pH. In some preferred embodiments, the pH is adjusted to be near the isoelectric point of the protein. Preferably, the protein is a vegetable protein. More preferably, the protein is a soy protein. The unfolding agent is preferably selected from the group consisting of ionic compounds, detergents, salts, and reducing agents. Preferred ionic compounds are selected from the group consisting of NaHSO₃, NaCl, p-hydroxybenzoic acid ethyl ester, p-hydroxybenzoic acid methyl ester, Na₂S, dehydroacetic acid, n-propyl p-hydroxybenzoate, and combinations thereof, more preferably NaHSO₃, NaCl, and combinations thereof, and is most preferably NaHSO₃ and/or a 1:1 mixture of NaHSO₃ and NaCl.

In yet another preferred embodiment of the present invention, a method of making a SAIPP as an adhesive in accordance with the present invention is provided. Generally, the method includes the step of adjusting the pH of a protein adhesive to be near the isoelectric point of the protein of the adhesive.

In another preferred embodiment of the present invention, a method of making SAIPP as an adhesive is provided. Generally, the method comprises the following steps: To begin, a first ionic compound is added to a mixture of protein and water. Then, the pH of the resulting solution is adjusted to be between about 6.0 and about 8.0. The mixture is then stirred and a second ionic compound is added to the mixture. The pH of the mixture is then adjusted to be near the isoelectric point of the protein. Following the pH adjustment, the mixture is stored for a period of time between about 12 hours and about 36 hours at a temperature between about 2° C. and about 20° C. Finally, the mixture is centrifuged to produce a suitable latex-like adhesive. Preferably, the protein for this method is a vegetable protein and more preferably, the protein is a soy protein. The first ionic compound is preferably any unfolding agent. The second ionic compound is preferably selected from the group consisting of NaHSO₃, NaCl, p-hydroxybenzoic acid ethyl ester, p-hydroxybenzoic acid methyl ester, Na₂S, dehydroacetic acid, n-propyl p-hydroxybenzoate, and combinations thereof, more preferably NaHSO₃, NaCl, and combinations thereof, and most preferably NaHSO₃ and/or a 1:1 mixture of NaHSO₃ and NaCl.

In yet another preferred embodiment of the present invention, another method of making SAIPP as an adhesive is provided. Generally, this method comprises the following steps: First, a mixture of protein and water with a basic pH is stirred. Next, an ionic compound is added to the mixture, and the pH of the mixture is thereafter returned to a basic pH, preferably to the same basic pH as in the first step. The mixture is then stirred again, after which the pH is adjusted to be near the isoelectric point of the protein, and the mixture is then centrifuged to produce a suitable latex-like adhesive. Preferably, the protein is a vegetable protein. More preferably, the protein is a soy protein. The ionic compound is preferably selected from the group consisting of NaHSO₃, NaCl, p-hydroxybenzoic acid ethyl ester, p-hydroxybenzoic acid methyl ester, Na₂S, dehydroacetic acid, n-propyl p-hydroxybenzoate, and combinations thereof, more preferably NaHSO₃, NaCl, and combinations thereof, and most preferably NaHSO₃ and/or a 1:1 mixture of NaHSO₃ and NaCl.

In a more preferred embodiment of the present invention, SAIPP as a soy latex-like adhesive using a soy protein isolate was produced in the following manner. Soy flour was preferably dissolved in distilled water or tap water, preferably distilled water, such that the concentration of the soy flour was preferably in the range of from about 2% to about 20%, more preferably from about 5% to about 15%, and was most preferably about 6%. Next, 2-mercapto-ethanol (“2ME”) was added to the soy-flour mixture at a ratio of about 0.001 mM/50 g soy flour to about 0.10 nM/50 g soy flour, more preferably at a ratio of about 0.005 mM/50 g soy flour to about 0.05 mM/50 g soy flour and most preferably at a ratio of about 0.01 mM/50 g soy flour to about 0.02 mM/50 g soy flour. The mixture was then stirred to homogenize the 2ME in the solution. The pH of the solution was then adjusted with 1M NaOH to be between about 6.0 and about 8.0, more preferably to between about 7.0 and about 8.0, and most preferably to about 7.6. The solution was then stirred for about 5 to about 240 minutes, more preferably for about 100 to about 200 minutes, and most preferably for about 120 minutes. The solution was then centrifuged to remove carbohydrates at a temperature in the range of up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The solution was centrifuged for about 2 to about 30 minutes, more preferably for about 10 to about 25 minutes, and most preferably for about 20 minutes. The pellet (“Pellet 1”) was then discarded. A chemical selected from the group consisting of NaHSO₃, NaCl, p-hydroxybenzoic acid ethyl ester, p-hydroxybenzoic acid methyl ester, Na₂S, dehydroacetic acid, n-propyl p-hydroxybenzoate, and combinations thereof, preferably NaHSO₃, NaCl, or combinations thereof, most preferably NaHSO₃ or a 1:1 mixture of NaHSO₃ and NaCl was then added to the supernatant (“Supernatant 1”) to a concentration between about 0.8 g/L to about 1.2 g/L based on the supernatant solution, more preferably between about 0.9 g/L to about 1.1 g/L, and most preferably to a concentration of about 1.01 g/L. Supernatant 1 was then stirred to homogenize the chemical. The pH of Supernatant 1 was then adjusted to be between about 4.0 to about 5.0, more preferably between about 4.2 and about 4.6, and most preferably to about 4.5 using 2N HCl. Supernatant 1 was then stirred again for a few minutes. Supernatant 1 was then stored at a temperature from about 2° C. to about 20° C., more preferably from about 3° C. to about 110° C., and most preferably at about 4° C. for about 12 hours to about 36 hours, more preferably for about 24 hours. After storage, Supernatant 1 was then centrifuged at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The solution was centrifuged for about 2 to about 30 minutes, more preferably for about 10 to about 25 minutes, and most preferably for about 20 minutes. The resulting supernatant (“Supernatant 2”) was then discarded, and the resulting pellet (“Pellet 2”) was a suitable latex-like adhesive in accordance with the present invention.

In another example, SAIPP can be made by directly dissolved soy proteins in water containing unfolding agents. Solid content can be from 1-40%. For the lower solid content solutions (about 1-15%), water can be removed by centrifuge or evaporation. For example, solid protein was dissolved in 1% SDS at a 12% solids content, water was evaporated at room temperature and condensed to a 40% solids content. The resultant was a clear, sticky adhesive.

In another preferred embodiment of the present invention, SAIPP as a soy latex-like adhesive was produced in the following manner. A soy protein was prepared, preferably by dissolving soy flour in distilled water or tap water, more preferably distilled water, such that the concentration of the soy flour was preferably in the range of from about 2% to about 20%, more preferably from about 5% to about 15%, and was most preferably about 6%. Next, 2ME was added to the soy-flour mixture at a ratio of about 0.001 mM/50 g soy flour to about 0.10 mM/50 g soy flour, more preferably at a ratio of about 0.005 mM/50 g soy flour to about 0.05 mM/50 g soy flour and most preferably at a ratio of about 0.01 mM/50 g soy flour to about 0.02 mM/50 g soy flour. The mixture was then stirred to homogenize the 2ME in the solution. The pH of the solution was then adjusted with 1M NaOH to be between about 6.0 and about 8.0, more preferably to between about 7.0 and about 8.0, and most preferably to about 7.6. The solution was then stirred for about 5 to about 240 minutes, more preferably for about 100 to about 200 minutes, and most preferably for about 120 minutes. The solution was then centrifuged to remove carbohydrates at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The solution was centrifuged for about 2 to about 30 minutes, more preferably for about 10 to about 25 minutes, and most preferably for about 20 minutes. The pellet (“Pellet 3”) was then discarded. A chemical selected from the group consisting of NaHSO₃, NaCl, p-hydroxybenzoic acid ethyl ester, p-hydroxybenzoic acid methyl ester, Na₂S, dehydroacetic acid, n-propyl p-hydroxybenzoate, and combinations thereof, preferably NaHSO₃, NaCl, or combinations thereof, most preferably NaHSO₃ or a 1:1 mixture of NaHSO₃ and NaCl was then added to the supernatant (“Supernatant 3”) to a concentration between about 0.8 g/L to about 1.2 g/L based on the supernatant solution, more preferably between about 0.9 g/L to about 1.1 g/L, and most preferably to a concentration of about 1.01 g/L. Supernatant 3 was then stirred to homogenize the chemical. The pH of Supernatant 3 was then adjusted to be between about 5.8 to about 6.8, more preferably between about 6.2 and about 6.6, and most preferably to about 6.4 using 2N HCl. Supernatant 3 was then stirred again for a few minutes. Supernatant 3 was then stored at a temperature from about 2° C. to about 20° C., more preferably from about 3° C. to about 10° C., and most preferably at about 4° C. for about 12 hours to about 36 hours, more preferably for about 24 hours. After storage, Supernatant 3 was then centrifuged at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The solution was centrifuged for about 2 to about 30 minutes, more preferably for about 10 to about 25 minutes, and most preferably for about 20 minutes. The resulting supernatant (“Supernatant 4”) was then discarded, and the resulting pellet (“Pellet 4”) was a suitable latex-like adhesive in accordance with the present invention.

In another preferred embodiment of the present invention, SAIPP as a soy latex-like adhesive was produced in the following manner. A soy protein was prepared, preferably by dissolving soy flour in distilled water or tap water, more preferably distilled water, such that the concentration of the soy flour was preferably in the range of from about 2% to about 20%, more preferably from about 5% to about 15%, and was most preferably about 6%. Next, 2ME was added to the soy-flour mixture at a ratio of about 0.001 mM/50 g soy flour to about 0.10 mM/50 g soy flour, more preferably at a ratio of about 0.005 mM/50 g soy flour to about 0.05 mM/50 g soy flour and most preferably at a ratio of about 0.01 mM/50 g soy flour to about 0.02 mM/50 g soy flour. The mixture was then stirred to homogenize the 2ME in the solution. The pH of the solution was then adjusted with 1M NaOH to be between about 6.0 and about 8.0, more preferably to between about 7.0 and about 8.0, and most preferably to about 7.6. The solution was then stirred for about 5 to about 240 minutes, more preferably for about 100 to about 200 minutes, and most preferably for about 120 minutes. The solution was then centrifuged to remove carbohydrates at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The solution was centrifuged for about 2 to about 30 minutes, more preferably for about 10 to about 25 minutes, and most preferably for about 20 minutes. The pellet (“Pellet 5”) was then discarded. A chemical selected from the group consisting of NaHSO₃, NaCl, p-hydroxybenzoic acid ethyl ester, p-hydroxybenzoic acid methyl ester, Na₂S, dehydroacetic acid, n-propyl p-hydroxybenzoate, and combinations thereof, preferably NaHSO₃, NaCl, or combinations thereof, most preferably NaHSO₃ or a 1:1 mixture of NaHSO₃ and NaCl was then added to the supernatant (“Supernatant 5”) to a concentration between about 0.8 g/L to about 1.2 g/L, more preferably between about 0.9 g/L to about 1.1 g/L based on the supernatant solution, and most preferably to a concentration of about 1.01 g/L. Supernatant 5 was then stirred to homogenize the chemical. The pH of Supernatant 5 was then adjusted to be between about 5.8 to about 6.8, more preferably between about 6.2 to about 6.6, and most preferably to about 6.4 using 2N HCl. Supernatant 5 was then stirred again for a few minutes. Supernatant 5 was then stored at a temperature from about 2° C. to about 20° C., more preferably from about 3° C. to about 10° C., and most preferably at about 4° C. for about 12 hours to about 36 hours, more preferably for about 24 hours. After storage, Supernatant 5 was then centrifuged to remove glycinin at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The solution was centrifuged for about 2 to about 30 minutes, more preferably for about 10 to about 25 minutes, and most preferably for about 20 minutes. The resulting pellet (“Pellet 6”) was then discarded. The resulting supernatant (“Supernatant 6”) was then adjusted with NaCl to a concentration in the range of from about 0.10 g/L to about 0.50 g/L, again based on the supernatant solution, more preferably in the range of from about 0.20 g/L to about 0.40 g/L, and most preferably to about 0.25 g/L. The pH of Supernatant 6 was then adjusted to be between about 4.5 to about 5.5, more preferably between about 4.8 and about 5.2, and most preferably to about 5.0 with 2N HCl. Supernatant 6 was then stored at a temperature from about 2° C. to about 20° C., more preferably from about 3° C. to about 10° C., most preferably at about 4° C. for about 1 hours to about 3 hours, more preferably for about 2 hours. After storage, Supernatant 6 was then centrifuged to remove residual glycinin at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The solution was centrifuged for about 2 to about 30 minutes, more preferably for about 10 to about 25 minutes, and most preferably for about 20 minutes. The resulting pellet (“Pellet 7”) was then discarded. The supernatant (“Supernatant 7”) was then diluted with a volume of distilled water or tap water, more preferably distilled water, equal to about twice the volume of Supernatant 7 in order to reduce its ionic strength. The pH of the diluted Supernatant 7 was then adjusted to a pH between about 4.2 and about 5.2, more preferably between about 4.6 and about 5.0, and most preferably to about 4.8 with 2N HCl. Supernatant 7 was then stored at a temperature from about 2° C. to about 20° C., more preferably from about 3° C. to about 10° C., and most preferably at about 4° C. for about 12 hours to about 36 hours, and more preferably for about 24 hours. After storage, Supernatant 7 was then centrifuged at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The solution was centrifuged for about 2 to about 30 minutes, more preferably for about 10 to about 25 minutes, and most preferably for about 20 minutes. The resulting supernatant (“Supernatant 8”) was then discarded, and the resulting pellet (“Pellet 8”) was a suitable latex-like adhesive in accordance with the present invention.

In another preferred embodiment of the present invention, SAIPP as a soy latex-like adhesive was produced in the following manner. Soy flour was preferably dissolved in distilled water or tap water, more preferably distilled water, such that the concentration of the soy flour was preferably in the range of from about 2% to about 20%, more preferably from about 5% to about 15%, and was most preferably about 6%. A chemical selected from the group consisting of NaHSO₃, NaCl, p-hydroxybenzoic acid ethyl ester, p-hydroxybenzoic acid methyl ester, Na₂S, dehydroacetic acid, n-propyl p-hydroxybenzoate, and combinations thereof, preferably NaHSO₃, NaCl, or combinations thereof, most preferably NaHSO₃ or a 1:1 mixture of NaHSO₃ and NaCl was then added to the mixture such that the concentration of the chemical was preferably in the range of from about 2% to about 20%, more preferably from about 3% to about 15%, more preferably from about 4% to about 10%, still more preferably from about 5% to about 8%, and was most preferably about 6%. The mixture was then stirred to homogenize the solution. The pH of the solution was then adjusted with 1M NaOH to be between about 6.0 and about 8.0, more preferably to between about 7.0 and about 8.0, and most preferably to about 7.6. The solution was then stirred for about 5 to about 240 minutes, more preferably for about 100 to about 200 minutes, and most preferably for about 120 minutes. The solution was then centrifuged to remove carbohydrates at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The solution was centrifuged for about 2 to about 30 minutes, more preferably for about 10 to about 25 minutes, and most preferably for about 20 minutes. The pellet (“Pellet 9”) was then discarded. The pH of the resulting supernatant (“Supernatant 9”) was then adjusted to be between about 4.0 to about 5.0, more preferably between about 4.2 to about 4.6, and most preferably to about 4.5 using 2N HCl. Supernatant 9 was then stirred for a few minutes. Supernatant 9 was then stored at a temperature from about 2° C. to about 20° C., more preferably from about 3° C. to about 10° C., and most preferably at about 4° C. for about 12 hours to about 36 hours, and more preferably for about 24 hours. After storage, Supernatant 9 was then centrifuged at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The solution was centrifuged for about 2 to about 30 minutes, more preferably for about 10 to about 25 minutes, and most preferably for about 20 minutes. The resulting supernatant (“Supernatant 10”) was then discarded, and the resulting pellet (“Pellet 10”) was a suitable latex-like adhesive in accordance with the present invention.

In another preferred embodiment of the present invention, SAIPP as a soy latex-like adhesive was produced in the following manner. Soy flour was preferably dissolved in distilled water or tap water, more preferably distilled water, such that the concentration of the soy flour was preferably in the range of from about 2% to about 20%, more preferably from about 5% to about 15%, and was most preferably about 6%. A chemical selected from the group consisting of NaHSO₃, NaCl, p-hydroxybenzoic acid ethyl ester, p-hydroxybenzoic acid methyl ester, Na₂S, dehydroacetic acid, n-propyl p-hydroxybenzoate, and combinations thereof, preferably NaHSO₃, NaCl, or combinations thereof, most preferably NaHSO₃ or a 1:1 mixture of NaHSO₃ and NaCl was then added to the mixture such that the concentration of the chemical to the soy flour was preferably in the range of from about 2% to about 20%, more preferably from about 5% to about 15%, and was most preferably about 6%. The mixture was then stirred to homogenize the solution. The pH of the solution was then adjusted with 1M NaOH to be between about 6.0 and about 8.0, more preferably to between about 7.0 and about 8.0, and most preferably to about 7.6. The solution was then stirred for about 5 to about 240 minutes, more preferably for about 100 to about 200 minutes, and most preferably for about 120 minutes. The solution was then centrifuged to remove carbohydrates at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The solution was centrifuged for about 2 to about 30 minutes, more preferably for about 10 to about 25 minutes, and most preferably for about 20 minutes. The pellet (“Pellet 11”) was then discarded. The pH of the resulting supernatant (“Supernatant 11”) was then adjusted to be between about 5.8 to about 6.8, more preferably between about 6.2 to about 6.6, and most preferably to about 6.4 using 2N HCl. Supernatant 11 was then stirred for a few minutes. Supernatant 11 was then stored at a temperature from about 2° C. to about 20° C., more preferably from about 3° C. to about 10° C., still more preferably at about 4° C. for about 12 to about 36 hours, and most preferably for about 24 hours. After storage, Supernatant 11 was then centrifuged at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The solution was centrifuged for about 2 to about 30 minutes, more preferably for about 10 to about 25 minutes, and most preferably for about 20 minutes. The resulting supernatant (“Supernatant 12”) was then discarded, and the resulting pellet (“Pellet 12”) was a suitable latex-like adhesive in accordance with the present invention.

In another preferred embodiment of the present invention, SAIPP as a soy latex-like adhesive was produced in the following manner. Soy flour was preferably dissolved in distilled water or tap water, more preferably distilled water, such that the concentration of the soy flour was preferably in the range of from about 2% to about 20%, more preferably from about 5% to about 15%, and was most preferably about 6%. A chemical selected from the group consisting of NaHSO₃, NaCl, p-hydroxybenzoic acid ethyl ester, p-hydroxybenzoic acid methyl ester, Na₂S, dehydroacetic acid, n-propyl p-hydroxybenzoate, and combinations thereof, preferably NaHSO₃, NaCl, or combinations thereof, most preferably NaHSO₃ or a 1:1 mixture of NaHSO₃ and NaCl was then added to the mixture such that the concentration of the chemical to the soy flour was preferably in the range of from about 2% to about 20%, more preferably from about 5% to about 15%, and was most preferably about 6%. The mixture was then stirred to homogenize the solution. The pH of the solution was then adjusted with 1M NaOH to be between about 6.0 and about 8.0, more preferably to between about 7.0 and about 8.0, and most preferably to about 7.6. The solution was then stirred for about 5 to about 240 minutes, more preferably for about 100 to about 200 minutes, and most preferably for about 120 minutes. The solution was then centrifuged to remove carbohydrates at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The solution was centrifuged for about 2 to about 30 minutes, more preferably for about 10 to about 25 minutes, and most preferably for about 20 minutes. The pellet (“Pellet 13”) was then discarded. The pH of the resulting supernatant (“Supernatant 13”) was then adjusted to be between about 5.0 to about 7.0, more preferably between about 5.5 to about 6.5, and most preferably to about 6.4 using 2N HCl. Supernatant 13 was then stored at a temperature from about 2° C. to about 20° C., more preferably from about 3° C. to about 10° C., and most preferably at about 4° C. for about 12 hours to about 36 hours, more preferably for about 24 hours. After storage, Supernatant 13 was then centrifuged to remove glycinin at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The solution was centrifuged for about 2 to about 30 minutes, more preferably for about 10 to about 25 minutes, and most preferably for about 20 minutes. The resulting pellet (“Pellet 14”) was then discarded. The pH of the supernatant (“Supernatant 14”) was adjusted to be between about 4.0 to about 6.0, more preferably between about 4.5 to about 5.5, and most preferably about 5.1 with 2N HCl. Supernatant 14 was then stirred for about 5 to about 20 minutes, more preferably for about 8 to about 15 minutes, and most preferably for about 10 minutes. Supernatant 14 was then stored at a temperature from about 2° C. to about 20° C., more preferably from about 3° C. to about 1° C., most preferably at about 4° C. for about 1 hours to about 3 hours, more preferably for about 2 hours. After storage, Supernatant 14 was then centrifuged to remove residual glycinin at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The solution was centrifuged for about 2 to about 30 minutes, more preferably for about 10 to about 25 minutes, and most preferably for about 20 minutes. The resulting pellet (“Pellet 15”) was discarded. The supernatant (“Supernatant 15”) was then diluted with a volume of distilled water or tap water, more preferably distilled water, equal to about twice that of Supernatant 15. The pH of the diluted Supernatant 15 was then adjusted to a pH between about 4.5 and about 5.5, more preferably between about 4.6 and about 5.0, most preferably to about 4.8 with 2N HCl. Supernatant 15 was then either not stored at all, or it was stored at a temperature from about 2° C. to about 20° C., more preferably from about 3° C. to about 10° C., and most preferably at about 4° C. for about 12 hours to about 36 hours, more preferably for about 24 hours. Either immediately after dilution or after being stored, preferably after being stored, Supernatant 15 was centrifuged at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The solution was centrifuged for about 2 to about 30 minutes, more preferably for about 10 to about 25 minutes, and most preferably for about 20 minutes. The resulting supernatant (“Supernatant 16”) was then discarded, and the resulting pellet (“Pellet 16”) was a suitable latex-like adhesive in accordance with the present invention.

In another preferred embodiment of the present invention, SAIPP as a soy latex-like adhesive was produced in the following manner. Soy flour was preferably dissolved in distilled water or tap water, more preferably distilled water, such that the concentration of the soy flour was preferably in the range of from about 2% to about 20%, more preferably from about 5% to about 15%, and was most preferably about 6%. The soy flour-mixture was then stirred until it was homogenized. The pH of the soy-flour mixture was then adjusted with 1M NaOH to be between about 6.0 and about 8.0, more preferably to between about 7.0 and about 8.0, and most preferably to about 7.6. The soy-flour mixture was then stirred for about 5 minutes to about 240 minutes, preferably for about 60 minutes to about 180 minutes, and most preferably for about 120 minutes. The soy-flour mixture was then centrifuged to remove carbohydrates at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The soy-flour mixture was centrifuged for about 2 to about 30 minutes, more preferably for about 10 to about 25 minutes, and most preferably for about 20 minutes. The resulting pellet (“Pellet 17”) was then discarded. The pH of the supernatant (“Supernatant 17”) was then adjusted with 2N HCl to a pH between about 5.5 and about 7.0, preferably between about 5.8 and about 6.8, and most preferably to about 6.4. Supernatant 18 was then stirred for a few minutes. Supernatant 18 was then stored at a temperature from about 2° C. to about 20° C., more preferably from about 3° C. to about 10° C., and most preferably at about 4° C. for about 12 hours to about 36 hours, more preferably for about 24 hours. After storage, Supernatant 18 was then centrifuged to remove glycinin at a temperature in the range of up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The solution was centrifuged for about 2 to about 30 minutes, more preferably for about 10 to about 25 minutes, and most preferably for about 20 minutes. The pellet (“Pellet 18”) was then discarded. The pH of the Supernatant (“Supernatant 18”) was then adjusted with 2N HCl to about 4.0 to about 6.0, preferably about 4.5 to about 5.5, and most preferably to about 4.8. Supernatant 18 was then stored was then stored at a temperature from about 2° C. to about 20° C., more preferably from about 3° C. to about 110° C., and most preferably at about 4° C. for about 12 hours to about 36 hours, more preferably for about 24 hours. After storage, Supernatant 18 was then centrifuged to remove glycinin at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The solution was centrifuged for about 2 to about 30 minutes, more preferably for about 10 to about 25 minutes, and most preferably for about 20 minutes. The resulting pellet (“Pellet 19”) was then freeze-dried and ground into powder to produce conglycinin protein powder. The conglycinin protein powder was dissolved in tap or distilled water, preferably distilled water, such that the concentration of the conglycinin was between about 25 to about 50% solid content, preferably between about 30 to about 45% solid content, and most preferably about 40% solid content and then generally stirred. A solution of a chemical selected from the group consisting of NaHSO₃, NaCl, p-hydroxybenzoic acid ethyl ester, p-hydroxybenzoic acid methyl ester, Na₂S, dehydroacetic acid, n-propyl p-hydroxybenzoate, and combinations thereof, preferably NaHSO₃, NaCl, or combinations thereof, most preferably NaHSO₃ or a 1:1 mixture of NaHSO₃ and NaCl was added to the protein solution such that the total concentration of the chemical was between about 1% to about 10%, preferably between about 2% and 5%, and most preferably about 3%. The mixture was then stirred for about 30 minutes or until it became cohesive. The result was a suitable latex-like adhesive in accordance with the present invention.

In another preferred embodiment of the present invention, SAIPP as a soy latex-like adhesive was produced in the following manner. Soy flour was preferably dissolved in distilled water or tap water, more preferably distilled water, such that the concentration of the soy flour was preferably in the range of from about 2% to about 20%, more preferably from about 5% to about 15%, and was most preferably about 6%. The solution was then stirred to obtain a homogeneous slurry. The pH of the slurry was adjusted to be between about 7.0 and about 9.0, preferably between about 7.5 and about 8.5, and most preferably to be about 8.0. The slurry was then centrifuged to remove carbohydrates at a temperature in the range of from up to about 50° C., more preferably from about 10° C. to about 30° C., and most preferably at about room temperature about 19° C. to about 26° C.). The force of centrifugation was between about 500 g to about 10,000 g, more preferably from about 10,000 g to about 15,000 g, and was most preferably about 2,000 g. The solution was centrifuged for about 2 to about 30 minutes, more preferably for about 10 to about 25 minutes, and most preferably for about 20 minutes. The resulting pellet “Pellet 20”) was discarded. The pH of the supernatant (“Supernatant 20”) was then adjusted to be between about 4.0 to about 6.0, more preferably to be between about 4.5 and about 5.5, and most preferably to be about 5.4. Supernatant 20 was then centrifuged to remove glycinin proteins at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The solution was centrifuged for about 2 to about 30 minutes, more preferably for about 10 to about 25 minutes, and most preferably for about 20 minutes. The resulting pellet (“Pellet 21”) was then discarded. The pH of the supernatant (“Supernatant 21”) was then adjusted to be between about 4.0 to about 6.0, more preferably to be between about 4.5 and about 5.5, and most preferably to be about 4.8, and then stirred for about 15 to about 75 minutes, preferably for about 30 to about 60 minutes. Supernatant 21 was then centrifuged to precipitate conglycinin at a temperature in the range of up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 12,000 g. The resulting supernatant (“Supernatant 22”) was then discarded. A chemical selected from the group consisting of NaHSO₃, NaCl, p-hydroxybenzoic acid ethyl ester, p-hydroxybenzoic acid methyl ester, Na₂S, dehydroacetic acid, n-propyl p-hydroxybenzoate, and combinations thereof, preferably NaHSO₃, NaCl, or combinations thereof, most preferably NaHSO₃ or a 1:1 mixture of NaHSO₃ and NaCl was added to the pellet (“Pellet 22”) in an amount equal to about 0.05% to about 0.5% by weight of Pellet 22, more preferably about 0.1% to about 0.4% by weight of Pellet 22, and most preferably 0.2% to about 0.3% by weight of Pellet 22. Pellet 22 and the chemical were then stirred for about 5 to about 30 minutes, preferably about 15 minutes, resulting in a suitable latex-like adhesive in accordance with the present invention.

In another preferred embodiment of the present invention, SAIPP as a soy latex-like adhesive was produced in the following manner. Soy flour, with a protein dispersion index between about 80 and about 100, more preferably between about 85 and about 95, and most preferably about 90, was preferably dissolved in distilled water or tap water, more preferably distilled water, such that the concentration of the soy flour was preferably in the range of from about 2% to about 20%, more preferably from about 5% to about 15%, and was most preferably about 5.5%. The pH of the soy protein was preferably modified to be between about 8.0 to about 11.0, more preferably about 9.0 to about 10.0, still more preferably about 9.3 to about 10.0, and most preferably to be about 9.5 using 2N NaOH. The soy protein solution was then stirred at about room temperature for preferably about 5 to about 100 minutes, more preferably about 30 to about 80 minutes, and most preferably for about 60 minutes. After stirring, a solid particulate, water-soluble ionic compound was added to the soy protein solution. The ionic compound was preferably selected from the group consisting of NaHSO₃, NaCl, NaOH, p-hydroxybenzoic acid ethyl ester, p-hydroxybenzoic acid methyl ester, Na₂S, dehydroacetic acid, n-propyl p-hydroxybenzoate, and combinations thereof, preferably NaHSO₃, NaCl, or combinations thereof, still more preferably NaHSO₃ or a 1:1 mixture of NaHSO₃ and NaCl, and was most preferably NaHSO₃. The amount of ionic compound added to the soy protein solution was preferably in the range of from about 0.5 g/L to about 10 g/L, more preferably from about 3 g/L to about 8 g/L, and most preferably was about 6 g/L. The pH of the resulting solution was then preferably adjusted at about room temperature to be between about 8.0 to about 11.0, more preferably about 9.0 to about 10.0, still more preferably about 9.5 to about 10.0, and most preferably to be about 9.5. The solution was then stirred at about room temperature for about 5 to about 240 minutes, more preferably for about 100 to about 200 minutes, and most preferably for about 120 minutes. The solution was then centrifuged to precipitate the pectin out at a temperature in the range of from up to about 50° C., more preferably from about 10° C. to about 30° C., and most preferably at about room temperature (about 19° C. to about 26° C.). The force of centrifugation was between about 200 g to about 1,000 g, more preferably from about 400 g to about 800 g, and was most preferably about 500 g. The solution was centrifuged for about 2 to about 30 minutes, more preferably for about 5 to about 20 minutes, and most preferably for about 10 minutes. The resulting pellet (“Pellet 23”) was discarded. The pH of the resulting supernatant (“Supernatant 23”) was then adjusted to be between about 5.0 to about 6.0, more preferably between about 5.2 and about 5.6, and most preferably to about 5.4. Supernatant 23 was then centrifuged at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 13,000 g. The solution was centrifuged for about 2 to about 30 minutes, more preferably for about 5 to about 20 minutes, and most preferably for about 10 minutes. The pellet (“Pellet 24”) was then separated from the resulting supernatant (“Supernatant 24”). The pH of Supernatant 24 was then adjusted to be between about 4.5 to about 5.5, preferably between about 4.6 and about 5.0, and most preferably to about 4.8. Supernatant 24 was then centrifuged at a temperature in the range of up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 13,000 g. Supernatant 24 was centrifuged for about 2 to about 30 minutes, more preferably for about 5 to about 20 minutes, and most preferably for about 10 minutes. The resulting supernatant (“Supernatant 25”) was discarded, and the resulting pellet (“Pellet 25”) was then mixed with Pellet 24 in order to form a suitable latex-like adhesive in accordance with the present invention.

In another preferred embodiment of the present invention, SAIPP as a soy latex-like adhesive was produced in the following manner. Soy flour, with a protein dispersion index between about 80 and about 100, more preferably between about 85 and about 95, and most preferably about 90, was preferably dissolved in distilled water or tap water, more preferably distilled water, such that the concentration of the soy flour was preferably in the range of from about 2% to about 20%, more preferably from about 5% to about 15%, and was most preferably about 5.5%. The pH of the soy protein was preferably modified to be between about 8.0 to about 11.0, more preferably about 9.0 to about 10.0, and most preferably to be about 9.5. The soy protein solution was then stirred at about room temperature for preferably about 5 to about 100 minutes, more preferably about 30 to about 80 minutes, and most preferably for about 60 minutes. After stirring, a solid particulate, water-soluble ionic compound was added to the soy protein solution. The ionic compound was preferably selected from the group consisting of NaHSO₃, NaCl, NaOH, p-hydroxybenzoic acid ethyl ester, p-hydroxybenzoic acid methyl ester, Na₂S, dehydroacetic acid, n-propyl p-hydroxybenzoate, and combinations thereof, preferably NaHSO₃, NaCl, or combinations thereof, still more preferably NaHSO₃ or a 1:1 mixture of NaHSO₃ and NaCl, and was most preferably NaHSO₃. The amount of ionic compound added to the soy protein solution was preferably in the range of from about 0.5 g/L to about 10 g/L, more preferably from about 3 g/L to about 8 g/L, and most preferably was about 6 g/L. The pH of the resulting solution was then preferably adjusted at about room temperature to be between about 8.0 to about 11.0, more preferably about 9.0 to about 10.0, and most preferably to be about 9.5. The solution was again stirred at about room temperature for about 5 to about 240 minutes, more preferably for about 100 to about 200 minutes, and most preferably for about 120 minutes. The pH of the resulting solution was then adjusted to be between about 5.0 to about 6.0, more preferably between about 5.2 and about 5.6, and most preferably to about 5.4. The solution was then centrifuged at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 13,000 g. The solution was centrifuged for about 2 to about 30 minutes, more preferably for about 5 to about 20 minutes, and most preferably for about 10 minutes. The pellet (“Pellet 26”) was then separated from the resulting supernatant (“Supernatant 26”). Supernatant 26 then has its pH adjusted from about 5.4 so that it would be between about 4.0 to about 5.0, more preferably between about 4.2 to about 4.9, and most preferably to about 4.8. Supernatant 26 was then centrifuged at a temperature in the range of from up to about 50° C., more preferably from about 10° C. to about 30° C., and most preferably at about room temperature (about 19° C. to about 26° C.). The force of centrifugation was between about 500 g to about 20,000 g, more preferably from about 10,000 g to about 15,000 g, and was most preferably about 13,000 g. Supernatant 26 was centrifuged for about 2 to about 30 minutes, more preferably for about 10 to about 25 minutes, and most preferably for about 20 minutes. The resulting supernatant (“Supernatant 27”) was discarded, and the resulting pellet (“Pellet 27”) was a suitable latex-like adhesive in accordance with the present invention in accordance with the present invention.

Those of skill in the art will recognize that centrifugation at a lower force and/or for a shorter period of time than the preferred forces and times listed above will result in latex-like adhesives that have a lower viscosity than the latex-like adhesives noted above. For example, such lower-viscosity adhesives resulted from centrifuging Supernatant 26 at about 13,000 g for 10 minutes; from centrifuging Supernatant 26 at about 1,000 g for about 12 minutes; and from centrifuging Supernatant 26 at about 500 g for about 6 minutes.

All examples described above can be obtained by directly dissolving selected proteins at 1-40% solid content in water that contains unfolding agent. Alternatively, the unfolding agent can be pre-mixed with the selected protein in dry form. The dissolved solution containing both protein and unfolding agent can be used as it is or water can be removed by centrifuge or condensation or evaporation methods.

In order to test the compressive strength, at least a portion of one of the latex adhesives described in the previous paragraph (“low-viscosity adhesive”) was mixed with sand samples (Foseco, Cleveland, Ohio) such that there was about 0.1% to about 5% of low-viscosity adhesive present compared to the amount of sand present, more preferably between about 0.5% to about 2% of low-viscosity adhesive present compared to the amount of sand present, and most preferably about 1% of low-viscosity adhesive (on a dry basis) present compared to the amount of sand present. This mixture was then loaded into a cylinder mold under slight pressure. The dimensions of the cylinder were in accordance with Foundry Standard XIII for Core Tests (Testing and Grading Foundry Sands by American Foundrymen's Association), which were about a 2-inch diameter and about a 2-inch length. The mold was preferably composed of polytetrafluoroethylene suitable for microwave curing. Holes were punched in the cylinder to insure that water would be able to escape during curing. The mixture and the cylinder mold were then cured in a microwave (SAM-155, CEM Corporation, Matthews, N.C.) using 90% power for about 2 minutes. The cured mixture was then tested for compressive strength using an Instron machine (Instron Corporation, Canton, Mass.). The average compressive strength of the mixture was about 2.6 MPa.

The teachings and content of all references cited herein are expressly incorporated by reference. Additionally, the teachings and content of the following references are also incorporated by reference herein: Fukushima, Danji, 1968, Internal structure of 7S and 11S globulin molecules in soybean proteins, Cereal Chemistry, 45(3): 203-224; Thanh, Vu Huu and Kazuo Shibasaki, 1976, Major protein of soybean seeds: a straightforward fractionation and their characterization, J. of Agriculture and Food Chemistry, 24(6): 1117-1121; Nagano, T., M. Hirotsuka, H. Mori, K. Kohyama, and K. Nishinar, 1992, Dynamic viscoelastic study on the gelation of 7S globulin from soybeans, J. of Agriculture and Food Chemistry, 40: 941-944; Sun, X., H. Kim, and X. Mo, 1999, Plastic performance of soybean protein components, J. of American Oil Chemists Society, 76(1): 117-123; Adachi, M., Y. Takenaka, A. B. Gidamis, E. Mikami, and S. Utsumi. 2001. Crystal structure of soybean proglycinin AlaBlb homotrimer, J. Molecular Biology, 305: 291-305; and Soybean protein subunits structure and sequences, Protein Data Bank, April 2002; Sun, Xiuzhi, In Biobased Polymers and Composites, By R. P. Wool and X. S. Sun, Elsevier Academic Press, NY, 2005, Chapter 9, page 292; Wolf, J. W., J. Agriculture and Food Chemistry. 1970, 18, 969; Staswick, P. E., M. A. Hermodson, and N.C. Nielsen, J. boil. Chem., 1984, 259, 13431; Nielsen, N.C., In New protein Foods 5: Seed Storage proteins, Eds., A. M. Altshul and H. L. Wilcke, Academic Press, Orlando, Fla., 1985, p. 27; Thanh, V. H.; K., Biochim. Biophys. Acta., 1977, 490, 370; Kinsella, J. E., S. Damodaran, and B. German, New Protein Foods 5: Seed Storage proteins, Eds., A. M. Altshul and H. L. Wilcke, Academic Press, Orlando, Fla., 1985, p. 107; Thanh, V. H., K. Shibasaki, J. Agric. Food Chem. 1079, 27, 805; Wolf, W. J. Briggs, D. R. Archs Biochem. Biophys, 1958, 76, 377; Mori, T., T. Nakamura, and S. Utsumi, J. Food Sci. 1982, 47, 26; Kinsella, J. E. J. Am. Oil. Chem. Soc. 1979, 56, 242; Zhang, J. and X. Y. Liu, J. of Chemical Physics, 2003, 119(20), 10972; McNaught, A. D. and A. Wilkinson, IUPAC Compendium of Chemical Terminology 2nd Ed., Royal Society of Chemistry, Cambridge, UK. G.B. 50; 1996, 68, 977; Thanh, V. H.; Shibsaki, K. J. Agric. Food Chem. 1976, 24, 1117; Iwabuchi, S.; Yamauchi, F. J. Agric. Food Chem. 1987, 35, 200; Iwabuchi, S.; Yamauchi, F. J. Agric. Food Chem. 1987, 35, 205; and Laemmli, U. K. Nature 1970, 227, 680.

### BRIEF DESCRIPTION OF THE DRAWING FIGURES

FIG. 1A is a TEM image of a soy protein sample with 3% total protein content and treated with sodium bisulfite at a pH of 5.4 and an ionic strength of 0;

FIG. 1B is an enlarged point of interest from FIG. 1A;

FIG. 1C is an enlarged point of interest from FIG. 1A;

FIG. 1D is a TEM image of a soy protein sample with 3% total protein content and treated with 3 g/L sodium bisulfite at a pH of 5.4 and an ionic strength of 0.02883;

FIG. 1E is an enlarged TEM image of a typical spherical cluster after treatment with 3 g/L sodium bisulfite;

FIG. 1F is an enlarged TEM image of another typical spherical cluster after treatment with 3 g/L sodium bisulfite;

FIG. 1G is a TEM image of a soy protein sample with 3% total protein content and treated with 6 g/L sodium bisulfite at a pH of 5.4 and an ionic strength of 0.05766;

FIG. 1H is an enlarged TEM image of a typical spherical cluster after treatment with 6 g/L sodium bisulfite;

FIG. 1I is an enlarged TEM image of a typical spherical cluster after treatment with 6 g/L sodium bisulfite;

FIG. 1J is a TEM image of a soy protein sample with 3% total protein content and treated with 12 g/L sodium bisulfite at a pH of 5.4 and an ionic strength of 0.1153 ionic strength;

FIG. 1K is an enlarged TEM image of a typical aggregate attached with many smaller spherical clusters and smaller aggregates after treatment with 12 g/L sodium bisulfite;

FIG. 1L is an enlarged TEM image of a typical aggregate attached with many smaller spherical clusters and smaller aggregates after treatment with 12 g/L sodium bisulfite;

FIG. 2A is a TEM image of a soy protein sample with 3% total protein content and treated with sodium bisulfite at a pH of 4.8 and an ionic strength of 0;

FIG. 2B is an enlarged image of FIG. 2A;

FIG. 2C is a TEM image of a soy protein sample with 3% total protein content and treated with 3 g/L sodium bisulfite at a pH of 4.8 and an ionic strength of 0.02883 ionic strength;

FIG. 2D is an enlarged TEM image of a typical network complex after treatment with 3 g/L sodium bisulfite;

FIG. 2E is an enlarged TEM image of a typical network complex after treatment with 3 g/L sodium bisulfite;

FIG. 2F is a TEM image of a soy protein sample with 3% total protein content and treated with 6 g/L sodium bisulfite at a pH of 4.8 and an ionic strength of 0.05766;

FIG. 2G is an enlarged TEM image of a typical network complex after treatment with 6 g/L sodium bisulfite;

FIG. 2H is an enlarged TEM image of a typical network complex after treatment with 6 g/L sodium bisulfite;

FIG. 2I is a TEM image of a soy protein sample with 3% total protein content and treated with 12 g/L sodium bisulfite at a pH of 4.8 and an ionic strength of 0.1153;

FIG. 2J is an enlarged TEM image of a typical partially destroyed spherical cluster after treatment with 12 g/L sodium bisulfite;

FIG. 2K is an enlarged TEM images of a typical partially destroyed spherical cluster after treatment with 12 g/L sodium bisulfite;

FIG. 2L is an enlarged TEM image of a typical individual spherical cluster after treatment with 12 g/L sodium bisulfite;

FIG. 2M is an enlarged TEM image of typical coupled spherical clusters after treatment with 12 g/L sodium bisulfite;

FIG. 3A is an enlarged TEM of a chain-like network structure for proteins having 62% water content and after treatment with 6 g/L sodium bisulfite;

FIG. 3B is an enlarged TEM of a chain-like network structure for proteins having 62% water content and after treatment with 6 g/L sodium bisulfite;

FIG. 3C is an enlarged TEM of a chain-like network structure for proteins having 62% water content after treatment with 6 g/L sodium bisulfite;

FIG. 3D is an enlarged TEM of a chain-like network structure for proteins having 62% water content after treatment with 12 g/L sodium bisulfite;

FIG. 3E is an enlarged TEM of a chain-like network structure for proteins having 62% water content after treatment with 12 g/L sodium bisulfite;

FIG. 3F is an enlarged TEM of a chain-like network structure for proteins having 62% water content after treatment with 12 g/L sodium bisulfite;

FIG. 4A is a LSM image of a cured protein without any treatment in accordance with the present invention;

FIG. 4B is a LSM image of a cured protein after treatment with 3 g/L sodium bisulfite;

FIG. 4C is a LSM image of a cured protein after treatment with 6 g/L sodium bisulfite;

FIG. 4D is a LSM image of a cured protein after treatment with 12 g/L sodium bisulfite;

FIG. 4E is a LSM image of a cured protein after treatment with 12 g/L sodium bisulfite;

FIG. 5A is a LSM image of a cured protein after treatment with 12 g/L sodium bisulfite and viewed as a free drop with a small amount of pressure;

FIG. 5B is a LSM image of a cured protein after treatment with 12 g/L sodium bisulfite, wherein the protein is spread onto the film with about 1 mm of thickness.

FIG. 6A is a light microscopy image of SAIPP with 3% ESO before being soaked in water;

FIG. 6B is a light microscopy image of the SAIPP with 3% ESO after being soaked in water for 24 hours;

FIG. 6C is a light microscopy image of SAIPP with 3% ESO before being soaked in water; and

FIG. 6D is a light microscopy image of SAIPP with 3% ESO after being soaked in water.

### DETAILED DESCRIPTION OF THE PREFERRED EMBODIMENTS

The following examples set forth preferred adhesives and procedures in accordance with the present invention. It is to be understood, however, that these examples are provided by way of illustration only, and nothing therein should be deemed a limitation upon the overall scope of the invention.

### Example 1

This Example describes SAIPP as latex-like adhesives prepared from soy protein isolates containing 2-mercapto-ethanol.

### Adhesive Preparation

About 50 g of soy flour (Cargill) was added to 800 ml of distilled water (about 1:15 to 1:20 ratio) and stirred until the flour was completely dissolved. About 18 drops of 2-mercapto-ethanol (2ME) (at about 0.01 to 0.02 m-mole/50 g soy flour ratio) was added to the soy-flour mixture and stirred for a few minutes. The pH of the mixture was then adjusted to 7.6 by stirring in 1 N NaOH for 120 minutes. The mixture was centrifuged at 4° C. and 12,000 g for 20 minutes to remove all carbohydrates that had precipitated. NaHSO₃ (sodium bisulfite in solid form) was added to the supernatant at 1.01 g/L based on the supernatant solution, and the pH of the solution was adjusted to 4.5 by stirring in 2N HCl for a few minutes and then storing the solution at 4° C. for 24 hours. The sample was centrifuged at 4° C. and 12,000 g for 20 minutes. The supernatant was discarded, and the precipitation was saved as the ion treated soy protein adhesive.

### Adhesive Performance

The resulting adhesive had a light yellowish color and a strong odor due to the 2ME. It had a smooth hand feel and better flow properties as compared to the soy proteins without ion treatment. However, this adhesive was difficult to spread into a thin layer and had low wet-tacking properties. The curing speed was also low at room temperature.

### Example 2

This Example describes SAIPP in liquid forms as latex-like adhesives prepared from soy protein containing 2-mercapto-ethanol.

### Adhesive Preparation

About 50 g soy flour (Cargill) was added to 800 ml distill water (about 1:15 to 1:20 ratio) and stirred in general until the dry flour was completely dissolved. About 18 drops 2-mercapto-ethanol (2ME) (at about 0.01 to 0.02 mMole/50 g soy flour ratio) was added into the soy flour-water mixture and stirred for a few minutes. The pH of the mixture was then adjusted to 7.6 by stirring in 1 N NaOH for 120 minutes. The mixture was centrifuged at 4° C. and 12,000 g for 20 minutes to remove all carbohydrates that had precipitated. NaHSO₃ was added to the supernatant at 1.01 g/L based on the supernatant solution, and the pH of the solution was adjusted to 6.4 by stirring in 2 N HCl for a few minutes and then storing the solution at 4° C. for 24 hours. The sample was centrifuged at 4° C. and 12,000 g for 20 minutes. The supernatant was discarded, and the precipitation was saved as the ion treated soy protein glycinin adhesive.

### Adhesive Performance

The adhesive was a light yellowish color with a strong odor due to 2ME, and it had good flowability and cohesiveness compared to the ion treated soy protein adhesives prepared in Example 1. This adhesive was easy to spread into a thin layer, which was clear, shining, and color less. This adhesive cured at room temperature within a few minutes. No phase separation was observed between protein and water after the long period of storage at 4° C., and adhesive structure and performance also remained the same.

### Example 3

This Example describes SAIPP in liquid form as latex-like adhesives prepared from soy protein containing 2-mercapto-ethanol.

### Adhesive Preparation

The ionic strength of the discarded supernatant from Example 2 was further adjusted with NaCl at 0.25 g/L based on the supernatant solution, and then the pH of the mixture was adjusted to 5.0 using 2N HCl. The sample was stored at 4° C. for 2 hours and then centrifuged at 4° C. and 12,000 g for 20 minutes to remove all glycinin residual. The ionic strength of the sample was reduced by adding distilled water at twice the volume of the supernatant solution. The pH of the sample was adjusted to 4.8 with 2 N HCl and stored at 4° C. for 24 hours, and then the sample was centrifuged at 4° C., 12,000 g for 20 minutes. The precipitation was saved as the ion treated soy protein conglycinin adhesive.

### Adhesive Performance

The adhesive was a light yellowish color with a strong odor due to the 2ME and excellent flowability and cohesiveness. This adhesive was much easier to spread into a thin layer than the adhesive prepared in Example 2. This adhesive was clear, shining, and colorless and cured at room temperature within a few minutes. No phase separation was observed between protein and water after the long storage at 4° C., and adhesive structure and performance remained the same.

### Example 4

This Example describes SAIPP as latex-like adhesives prepared from soy protein isolate using sodium bisulfite.

### Adhesive Preparation

About 50 g of soy flour (Cargill) was added to 800 ml of distilled water (about 1:15 to 1:20 ratio) and was stirred until the flour completely dissolved. About 4.8 g NaHSO₃ (at about 6% based on soy flour solution by weight) was added to the soy flour-water mixture and stirred for a few minutes. The pH of the mixture was then adjusted to 7.6 by stirring in 1 N NaOH for 120 minutes. The mixture was centrifuged at 4° C., 12,000 g for 20 minutes to remove all carbohydrates that had precipitated. The pH of the supernatant was adjusted to 4.5 by stirring in 2 N HCl for a few minutes, and the supernatant was then stored at 4° C. for 24 hours. The sample was centrifuged at 4° C., 12,000 g for 20 minutes. The precipitation was saved as the ion treated soy protein adhesive.

### Adhesive Performance

The adhesive was a light yellowish color and odor free, and had smooth hand feel and better flow properties compared to the soy proteins without ion treatment. This adhesive had similar adhesion performance to the adhesive prepared in Example 1.

### Example 5

This Example describes SAIPP as latex-like adhesives prepared from soy protein using sodium bisulfite.

### Adhesive Preparation

About 50 g of soy flour (Cargill) were added to 800 ml distilled water (about 1:15 to 1:20 ratio) and stirred until the flour was completely dissolved. About 4.8 g of NaHSO₃ (about 6% based on soy flour solution by weight) was added to the soy flour-water mixture and stirred for a few minutes. The pH of the mixture was then adjusted to 7.6 by stirring in 1 N NaOH for 120 minutes. The mixture was centrifuged at 4° C., 12,000 g for 20 minutes to remove carbohydrates that had precipitated. The pH of the supernatant was adjusted to 6.4 by stirring in 2 N HCl for a few minutes, and the supernatant was then stored at 4° C. for 24 hours. The sample was again centrifuged at 4° C., 12,000 g for 20 minutes. The supernatant was discarded, and the precipitation was saved as the ion treated protein glycinin adhesives.

### Adhesive Performance

The adhesive was a light yellowish color and odor free, with a good flowability and cohesiveness compared to the ionic treated soy protein adhesives prepared in Examples 1 and 4. This adhesive was easy to spread into a thin layer, which was clear, shining, and colorless. This adhesive cured at room temperature within a few minutes. No phase separation was observed between protein and water after the long period of storage at 4° C., and adhesive structure and performance remained the same.

### Example 6

This Example describes SAIPP as latex-like adhesives prepared from soy protein using sodium bisulfite.

### Adhesive Preparation

The pH of the discarded supernatant in Example 5 was adjusted to 5.1 with 2 N HCl and stirred for 10 minutes. The sample was stored at 4° C. for 2 hours and then centrifuged at 4° C., 12,000 g for 20 minutes to remove all glycinin residual. The supernatant was diluted by adding distilled water at twice the volume of the supernatant. The pH of the diluted solution was adjusted to 4.8 with 2 N HCl, and the solution was then stored at 4° C. for 24 hours. The solution was centrifuged again at 4° C., 12,000 g for 20 minutes. The precipitation was saved as the ion treated soy protein conglycinin adhesive.

### Adhesive Performance

The adhesive was a light yellowish color and odor free. It had excellent flowability and cohesiveness. This adhesive was much easier to spread into a thin layer than the adhesive prepared in Example 5. This adhesive was clear, shining, and colorless and cured at room temperature within a few minutes. No phase separation was observed between protein and water after the long period of storage at 4° C., and adhesive structure and performance remained the same. The moisture content of the adhesive was about 40%.

### Example 7

This Example describes SAIPP as latex-like adhesives prepared from soy protein using sodium bisulfite.

### Adhesive Preparation

To avoid the 24 hour storage period and thus reduce processing time, the diluted supernatant with pH 4.8, prepared in Example 6, was directly centrifuged at 4° C., 12,000 g for 20 minutes. The precipitation was saved as the ion treated soy protein conglycinin adhesive.

### Adhesive Performance

The adhesive performed similar to the conglycinin protein (7S) extracted by the normal procedure. It was not cohesive and had poor wet-tack properties. The flow behavior of the adhesive was also poor as compared with the adhesive prepared in Example 6. The adhesive was also difficult to apply and took longer to cure.

### Example 8

This Example describes SAIPP as latex-like adhesives prepared from soy protein using sodium bisulfite.

### Adhesive Preparation

To reduce processing time, low moisture modification technology was applied. The conglycinin protein was prepared by following normal extraction procedures. About 2 g of the conglycinin protein powder was added into 3 ml distilled water (about 40% solid content) and stirred. Then about 1 ml of solution with about 12% NaHSO₃ was added to the mixture and stirred for about 30 minutes or until the mixture became cohesive. The sample was considered as the ion treated conglycinin protein adhesive.

### Adhesive Performance

The adhesive was cohesive, a yellowish color, and odor free. It contained foams that disappeared after storage at 4° C. for a few days. The adhesive was easy to apply and spread into a thin layer which was shining and colorless. The adhesive cured at room temperature within a few minutes. The flowability of the adhesive was tolerable, but not as good as the adhesive prepared in Example 6.

### Example 9

This Example describes the effects of storage temperature and time on adhesive performance.

### Test Specimen Preparation and Testing Methods

The adhesive prepared using Example 6 was stored at room temperature, at 23° C., and at −15° C. Adhesive was applied to two pieces of cherry wood samples. The testing specimen preparation, the testing procedures, and the evaluation methods followed the same procedures used in the lab.

Veneer cherry wood was used as an adherent provided by Veneer One (Oceanside, N.Y.). The dimensions of the wood samples were 50 mm×127 mm×3 mm. The prepared adhesives were applied to each end of two pieces of wood samples in about a 127 mm×20 mm area. The wood samples with adhesives were allowed to rest for 3 minutes at room temperature at about 50 to 60% relative humidity (RH) and then were pressed together at 190° C., 1.4 MPa for 5 minutes. The glued sample was preconditioned at 23° C. and 50% RH for 48 hours, was cut into five testing specimens, and then was preconditioned at 23° C. and 50% RH for another 5 days before testing.

Adhesive strength was determined using an Instron machine (Model 4465, Canton, Mass.) according to ASTM D2339. For the water resistance test, specimens were evaluated according to ASTM D1151 for effects of moisture and temperature on adhesive bonds and ASTM D1183 for resistance of adhesive to cyclic lab aging. Samples were soaked in tap water at 23° C. for 48 hours and were immediately tested for wet strength.

Boiling tests were conducted following PS1-95 method which included placing one group of specimens in a tank of boiling water separated by wire screens in such a manner that all surfaces were freely exposed to water. The specimens were forced to be immersed at least 51 mm deep during the boiling test cycle and stay immersed for 4 hours. The specimens were then dried for 20 h at 63±3° C. with sufficient air circulation to reduce the moisture content (MC) of the specimens to original, within an allowable variation of ±1% MC. The 4 hour boiling cycle was repeated, and the specimens were removed and cooled in running tap water at 18 to 27° C. for 1 hour. Then, the specimens were evaluated for wet strength.

### Adhesive Strength

The strength of the adhesives was not affected by storage temperature and time (see Table 1). For the dry tests, the cohesive wood failure (CWF) measured 100%. The CWF of the wet and boiling test samples were not evaluated. About 90% of the glued area showed coarse fibers for wet test samples, and very fine fibers were observed for boiling test samples.

The adhesive stored at room temperature covered with a cap remained mold free for several months. Therefore, shelf life of the adhesive should be not a problem. The NaHSO₃ has been used as a food preserver to extend shelf life at an FDA-approved level of less than 0.3%.

TABLE 1

| | | | |
|---|---|---|---|
| | Dry Strength | Wet Strength | Boiling Strength |
| Time of Storage | (MPa) | (MPa) | (MPa) |

Samples Stored at Room Temperature (23° C.)

| | | | |
|---|---|---|---|
| 0 months | 6.54 ± 1.20 | 4.35 ± 0.34 | 2.78 ± 0.06 |
| 1 month | 6.34 ± 1.12 | 4.25 ± 0.27 | 2.80 ± 0.14 |
| 2 months | 6.64 ± 1.56 | 3.89 ± 0.32 | 2.65 ± 0.35 |
| 4 months | 6.24 ± 1.65 | 3.69 ± 0.31 | 2.30 ± 0.49 |

Samples Stored at 4° C.

| | | | |
|---|---|---|---|
| 3 months | 6.30 ± 1.87 | 3.67 ± 0.41 | 2.64 ± 0.21 |

Samples Stored at −15° C. (Thawed at Room Temperature)

| | | | |
|---|---|---|---|
| 3 cycles | 5.5-6.5 ± 2.0 | 3.54 ± 0.60 | 2.33 ± 048 |

### Example 10

This Example describes the formation of SAIPP as latex-like adhesives using a short procedure. Based on the mechanism described in the conceptual section, both mechanical force and ionic strength should be provided to produce the latex adhesive from soy protein. For the long procedure, ionic strength was applied to the protein with excess water. In this experiment, the ionic strength was applied to protein at low moisture content. Once the protein structure becomes swollen and stretched outwards by ionic treatment, the protein becomes entangled under mechanical stirring force, forming viscous sticky semi fluid adhesive.

### Adhesive Preparation

Defatted soy flour was dissolved in distilled water at about 6% solid content and stirred for about 30 minutes or until a uniform slurry was formed. The pH of the slurry was adjusted to 8.0 and centrifuged to remove carbohydrates. The pH of the supernatant was adjusted to 5.4 and centrifuged to remove glycinin proteins (11S) as precipitate (both carbohydrate and 11S can be removed in one procedure depending on the end use of the carbohydrate and 11S). Then the pH of the supernatant was adjusted to 4.8, stirred for 30-60 minutes, and centrifuged. The NaHSO₃ solution or powder was added to the precipitate 7S protein at about 0.2 to 0.3% (by weight based on precipitate protein) and then stirred for 15 minutes at intensive mechanical shearing. The final moisture content of the adhesive was about 40%.

### Adhesive Performance

The adhesive obtained using the short procedure had similar adhesive strength compared to the adhesives from the previous examples. See Table 2 below for the results obtained. For the dry test, the cohesive wood failure (CWF) measured 100%. The CWF of wet and boiling test samples were not evaluated. About 90% of the glued area showed coarse fibers for wet test samples, and very fine fibers were observed for boiling test samples. However, the flowability of the adhesive was lower than that prepared using the long procedure. The structure of the adhesive from the short procedure was not as smooth as that with the long procedure, which might be caused by poor stretching and entanglement due to the short reaction time.

Three major advantages of using short procedure are that: 1) the processing time was significantly reduced (about 6 times), which means increased efficiency; 2) the short procedure allows for a lower amount of sodium bisulfate to be used (less than 0.3%) which is the FDA-approved level of a food preserver; and 3) the water discharged from the centrifuge is recyclable water containing no sodium bisulfate because the chemical is added after centrifugation. Thus, the process is more environmentally friendly.

TABLE 2

| Procedure | Dry Strength | Wet Strength | Boiling Strength |
|---|---|---|---|
| Followed | (MPa) | (MPa) | (MPa) |
| Long Procedure | 6.54 ± 1.20 | 4.35 ± 0.34 | 2.78 ± 0.06 |
| Short Procedure | 7.12 ± 0.39 | 3.54 ± 0.36 | 2.21 ± 0.45 |

### Example 11

This Example compares chemicals for SAIPP as latex-like soy adhesive processing. Sodium chloride (NaCl) was tested following the long procedure as described in Example 6. The adhesive performed similarly to that prepared using NaHSO₃, but had poor flowability. In addition, slight phase separation between water and protein was observed after a few days of storage, which became continuous after stirring.

The flowability and phase separation of the adhesive using NaCl were improved by adding a small amount of NaHSO₃ at 1:1 ratio (the total chemical amount remained the same). The adhesive strength using the short procedure is presented in Table 3. Other chemicals were also tried including p-hydroxybenzoic acid ethyl ester, p-hydroxybenzoic acid methyl ester, sodium sulfite, dehydroacetic acid, and n-propyl p-hydroxybenzoate. Adhesives prepared using these chemicals all had lower flowability and phase separation problem by observation, but no testing data were collected.

TABLE 3

| | Dry Strength | Wet Strength | Boiling Strength |
|---|---|---|---|
| Chemical Used | (MPa) | (MPa) | (MPa) |
| NaHSO₃ | 7.12 ± 0.39 | 3.54 ± 0.36 | 2.21 ± 0.45 |
| NaCl/NaHSO₃ | 6.45 ± 0.45 | 4.36 ± 0.43 | 2.83 ± 0.32 |

### Example 12

This Example describes short procedures for the production of SAIPP as latex-like soy adhesives from soy flour.

Commercial soy flour with a 90 protein dispersion index (PDI) provided by Cargill was used. The soy flour was dissolved in distilled water at a ratio of 1:16-20. First, the flour was mixed using small amount of water, and then the full amount of water was added, and the pH was adjusted to 9.5 by stirring in 2N NaOH solution at room temperature for 1 hour. The protein partially unfolded during this time because of the presence of NaOH.

Next, sodium bisulfite (NaHSO₃) was added into the flour slurry at 6 g/L at room temperature. The pH of the slurry was kept at about 9.5, and the slurry with NaHSO₃ was stirred for another 2 hours. Proteins interacted with NaHSO₃ and unfolded, resulting in more hydrophobic groups outwards. The pH of the slurry was adjusted to 5.4 and then centrifuged at 13000 g at 4 C for 10 minutes. The precipitation was discarded (mainly carbohydrates and 11S). The pH of the supernatant was adjusted from 5.4 to 4.8 using 2N HCl and centrifuged at 13000×g at 4C for 20 minutes. The precipitation was the latex-like adhesive and had a moisture content of about 37%. The adhesive performance was similar to the performance of adhesives obtained using the long procedure described in Example 4.

### Example 13

This Example describes short procedures for the production of low viscosity SAIPP as latex-like soy adhesives from soy flour.

For this example, the same procedure described in Example 12 was followed. The only difference being that the centrifuge time in the last step was 10 minutes instead of 20 minutes. The precipitation was the latex-like soy adhesive with a lower viscosity, having about 64% moisture content. The adhesive performance of this adhesive was similar to that obtained from Example 4 but needed longer time for curing due to the higher moisture content. Even lower viscosity adhesive can be obtained by using a shorter centrifuge times and lower centrifuge forces, such as 1000 g for 12 minutes or 500 g for 6 minutes at 4° C. The adhesives obtained using lower centrifuge intensity contained about 87% moisture content, and had less tack adhesive properties.

### Example 14

This Example describes short procedures for the high yield production of SAIPP as latex-like soy adhesives from soy flour.

Commercial soy flour with a protein dispersion index (PDI) of 90 (Cargill) was used. The soy flour was dissolved in distilled water at a ratio of 1:16-20. First, the flour was mixed using a small amount of water, and then full amount of water was added. The pH was adjusted to 9.5 by stirring in a 2N NaOH solution at room temperature for 1 hour. Next, sodium bisulfite (NaHSO₃) was added into the flour slurry at 6 g/L slurry at room temperature. The pH of the slurry should be kept or readjusted to maintain a pH between 9.5-10 (9.5). The slurry with NaHSO₃ was stirred for another 2 hours and then centrifuged at 4° C., 500 g for 10 minutes to precipitate the pectin out. The pH of the supernatant was adjusted to 5.4, and then centrifuged at 4° C., 13,000 g for 10 minutes to precipitate 11S, semicellulose and cellulose (precipitation II). The pH of the second supernatant was adjusted to 4.8 and centrifuged at 4° C., 13,000 g for 10 minutes to precipitate the 7S (precipitation III). Precipitations II and III were mixed uniformly, and the resulting mixture can be used as a latex-like adhesive with about 64% moisture content. Viscosity and tack property of this adhesive were lower than that from Example 4, but it still has potential applications, such as foundry, wood, labeling, wall paper adhesives, etc.

### Example 15

This Example describes microwave curing.

Adhesives produced using the procedure described in Example 13 were applied to sand samples. A 1% adhesive by solid (Foseco) was used to mix with the sand samples. The mixture was loaded into a cylinder mold made by the mechanical workshop at KSU. The dimensions of the cylinder followed the foundry standard XIII for core test (Testing and Grading Foundry Sands by American Foundrymen's Association) with a 2 inch diameter and a 2 inch length. The material of the mold was polytetrafluoroethylene (PTFE) suitable for microwave curing. Some holes were made in the cylinder and the cover of the mold to allow water to escape during the microwave curing. The coated sand sample was loaded with slight pressure into the mold and cured in a microwave (SAM-155, CEM Corporation, Matthews, N.C.) using 90% power for 2 minutes. The cured sample was compressively tested using Instron and had about 2.6 MPa average compressive strength.

### Example 16

This Example describes variable ranges tested for the present invention.

All plant proteins and animal proteins (preferred soy proteins, more preferred soy conglycinin proteins) can be raw materials to produce protein-based “latex” like adhesives in accordance with the present invention. Using soy flour as a starting material, for example, variable ranges for each step are given in the following flow chart.

Example of producing SAIPP as latex adhesives from soy flour

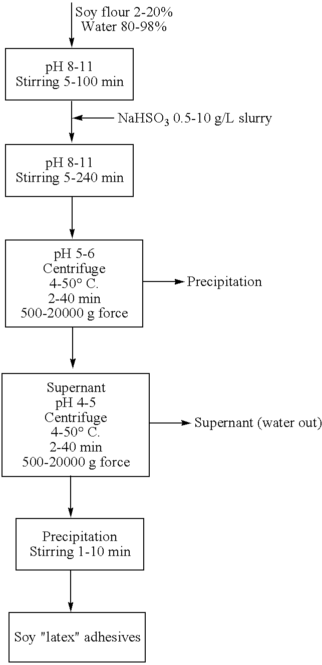

### Discussion

In general, molecular size, structure, unfolding degree, unfolding agent, and ionic strength should be important in SAIPP preparation. Protein structure, such as functional groups types and concentration, amino acid sequences and conformation, and protein molecular size, should be a major factor in determining unfolding agents and types such as ionic strength that can make proteins form complexes with desirable wet-tack features.

Mechanical force and water content are considered other important factors for protein complex formation. The adhesive prepared from Example 7 was not nearly as good as that from Example 6. The only difference was the 24 hour storage time. The swollen and unfolded proteins precipitated slowly along with those ions attracted to them during 24 hour storage. These precipitated proteins became structurally entangled during centrifugation because of the centrifugal force and formed a continuous complex that was stable and flowable. However, in Example 7, the swollen and unfolded conglycinin proteins suspended in the solution would precipitate upon centrifugation. The ions attracted to the proteins would remain in the solution because the centrifugal force was large enough to break the attractive force between ions and charged functional groups of proteins. The swollen and unfolded protein would fold up again back to its equilibrium state. The adhesives prepared in Example 8 had similar adhesive performance as those in Example 6 because the distances between molecules were small, and the swollen and unfolded proteins became entangled upon mechanical mixing. The adhesive prepared in Example 8 had lower flowability than that in Example 6. The ionic strength in Example 8 could not be strong enough to stretch proteins, or proteins could not be fully exposed to ions due to low moisture content and the foams generated in mixing, and or interaction time between proteins and ions might not be long enough. The performance of the adhesive prepared in Example 8 can be improved by optimizing processing procedures, water content, and ionic strength. Reducing water content by evaporation or condensation is another effective method for forming such unfolded protein complexes.

### Example 17

This Example produced SAIPP in accordance with the present invention and tested various aspects affecting SAIPP properties and characteristics.

Defatted soy flour, having a protein dispersion index of 90, was provided by Cargill (Cedar Rapids, Iowa). The soy flour contained about 50% protein content and about 10% moisture content with 98% of all particles being capable of passing through 100 mesh. A. C. S. certified sodium bisulfite (NaHSO₃) in solid form (Fisher Scientific) having 56.7% SO₂, 0.005% chloride, 0.0003% heavy metal (pb), 0.003% water insoluble, and 0.0005% iron, was provided. Ionic strength of the protein was estimated using the equation (Im=½ΣM_{B}Z_{B}²), based on a molality basis. Where the sum goes over all the ions B used in the system. Z_{B} is the charge number of ion B. In this Example, NaHSO₃ was used to prepare water solutions with three different ionic strength levels (0.02883, 0.05766, and 0.1153). The ions generated by adjusting the pH of the system using either sodium hydroxide (NaOH) or hydrochloride (HCl) were neglected.

To prepare the protein samples, the soy flour was dissolved in distilled water at 1:16 (soy flour:water) ratio at room temperature. The pH of the slurry was adjusted to 9.5. Sodium bisulfite was added to the soy flour slurry at various amounts to produce four different levels of ionic strength, 0.0, 0.02883, 0.05766, and 0.1153. The slurry was stirred at room temperature for two hours before being centrifuged (C I) at 12,000×g force for 10 minutes at room temperature to remove fibers. The pH of the supernatant was adjusted to 5.4 and 4.8 respectively and collected for low protein concentration samples (about 3% solid content). The protein samples contained about 39% glycinin and 23% conglycinin and other polypeptides components as estimated by using SDS-PAGE (Sodium Dodecyl Sulfate-Polyacrylamide Gel Electrophoresis) method. Next, the liquid sample having the pH of 5.4 was centrifuged (C II) at 10,000×g force for 10 minutes at room temperature. The precipitate of the centrifuge (C II) was mainly glycinin protein. The pH of the supernatant of the centrifuge (C II) was adjusted to 4.8 and was centrifuged (C III) at 10,000×g force for 10 min. The precipitates of the centrifuge (C II) were collected as high protein concentration samples (about 38% solid content). The precipitate from centrifuge C III was still a mixture of several polypeptides, but the ratio of conglycinin to glycinin was increased from 0.58 to 0.66. Some glycinin proteins were removed during centrifuge C III.

To prepare α and β polypeptides, conglycinin (7S) was purified by separating the crude 7S protein fractions from soy flour according to the method of Thanh and Shibasaki. The crude 7S fraction was purified by ammonium sulfate fraction as described by Iwabuchi and Yamauchi. The crude 7S fraction, at 3% concentration, was dissolved in phosphate buffer (2.6 mM KH₂PO₄, 32.5 mM K₂HPO₄, 0.4 M NaCl, 10 mM mercaptoethanol, 1 mM EDTA). Ammonium sulfate was added to the protein solution to 75% saturation. The precipitate was centrifuged off, and additional ammonium sulfate was added to the supernatant to 90% saturation. After centrifugation, the precipitate was collected and desalted using Centricon Plus-80 centrifugal filter (Millipore Corp., Bedford, Mass.).

The α and β polypeptides were isolated and purified using the anion exchange chromatography method. The purified 7S (3 g) was applied to a DEAE Sepharose Fast Flow column (2.6×40 cm) equilibrated with 20 mM Tris buffer (pH 7.5) containing 6M urea. Gradient elution was carried out with a linear increase of NaCl concentration from 0 to 0.4 M (800 ml each). Column effluents were collected in 15.6 ml-fractions at a flow rate of 1.3 ml/min. The collected fractions were analyzed using SDS-PAGE. Fractions containing α and β polypeptides were pooled and dialysis performed against deionized water, respectively, and freeze dried and collected as the α and β polypeptide samples.

A Transmission Electron Microscope (TEM), Model CM 100 (FEI Company, Hillsboro, Oreg., USA), was operated at 100 kV. Protein samples were diluted to 1% with distilled water and sonicated for 3 minutes in an L&R 320 Ultrasonic (L&R Manufacturing Company, Keary, N.J., USA), before being absorbed for approximately 30 seconds at room temperature onto Formvar/Carbon coated 200 mesh copper rids (Electron Microscopy Sciences, Fort Washington, Pa., USA), stained with 2% (w/v) uranyl acetate (Ladd Research Industries, Inc., Burlington, Vt., USA) for 60 seconds at room temperate before being viewed by TEM.

Light Microscopy (LSM) images were taken on an Axioplan 2 MOT Research Microscope (Carl Zeiss, Inc., Thomwood, N.Y., USA) equipped with a Zeiss Axiocam HR digital camera, a fully motorized stage with mark and find software, Plan Neofluor objectives (1.25x/0.035, 10x/0.3, 20x/0.5, 40x/0.75, 40x/1.3 oil), Plan apochromat objectives (63x/1.4 oil, 100x/1.4 oil), an Achroplan objective (4x/0.1), differential contrast interference (DIC), phase contrast (ph), dark field, bright field, and Axiovision 3.1 software with interactive measurements and 3D deconvolution modules.

A small drop of a fresh protein sample obtained from the centrifuge CIII was made onto a piece of glass (Fisher Scientific). DIC images were taken at various scales before, during, and after curing of the protein sample. Fluorescence images were analyzed as needed for some samples.

To estimate the composition of the polypeptides of the invention, SDS-PAGE was performed using a discontinous buffer system on a 12% separating gel and 4% stacking gel as described by Laemmli. Protein samples were mixed with SDS-PAGE sample buffer solution containing 5% b-mercaptoethanol, 2% SDS, 25% glycerol, and 0.01% bromphenol blue. Each well was loaded with approximately 5 μg of protein sample. The gel electrophoresis was carried out at 100V, constant voltage. The gel was stained with 0.25% Coomassie Brilliant Blue-R250 and destained with a solution containing 10% acetic acid and 40% methanol. Molecular weight marker proteins were run along with the samples. The percentage of polypeptide component was estimated by analyzing the gel image with Kodak 1D Image analysis software, version 4.6 (Eastman Kodak Company, Rochester N.Y.).

The protein samples were then subjected to rheology analysis. Differential scanning calorimetry was performed using a Perkin-Elmer Pyris-1 Differential Scanning Calorimeter (DSC) (Perkin-Elmer, Norwalk, Conn.) in order to study thermal transitions of the protein samples. The instrument was calibrated with indium and zinc standards before measurements, and all measurements were conducted under a nitrogen atmosphere. The sample was sealed in a large-volume stainless-steel DSC pan with an O-ring, thereby preventing any water loss during the DSC scan. All samples were held at 25° C. for 1 minute and then were scanned to 220° C. at 10° C./minute increments. After that, the samples were quenched to 25° C., held for 1 minute, and scanned again to 220° C. at 10° C./minute. The denaturation temperature (T_{d}), enthalpy of denaturation (ΔH_{d}), and glass transition temperature (T_{g}) were obtained from the first scan.

An X-ray scattering method was used to determine crystal and amorphous phases of the protein samples. A Philips APD 3520 wide angle X-ray Diffractometer was used. A voltage of 35 kV, a current of 20 mA, and curved crystal graphite monochromator (λ=0.154 μm) were employed. The protein samples were then freeze-dried and ground into powder. The powder sample was continuously scanned from 10° to 35° (2θ) with a speed of 2° (2θ)/minute.

### Results

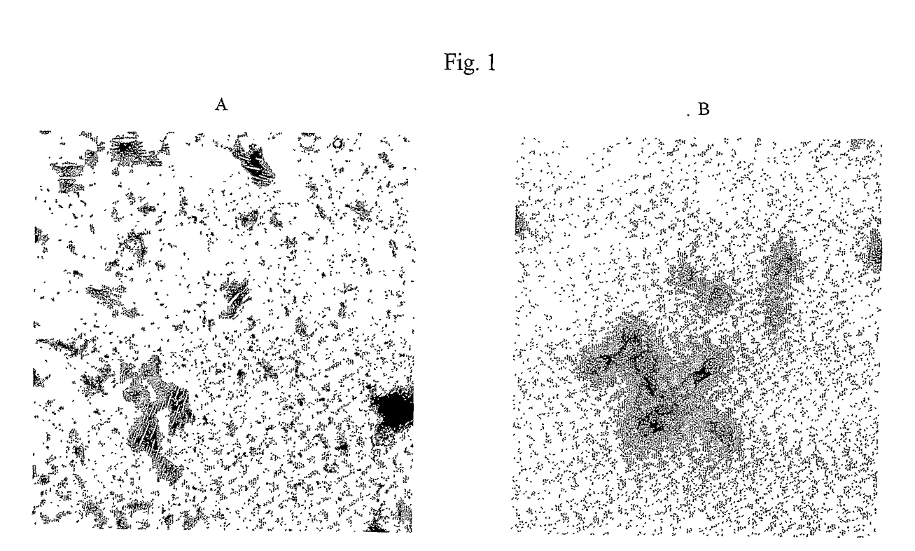

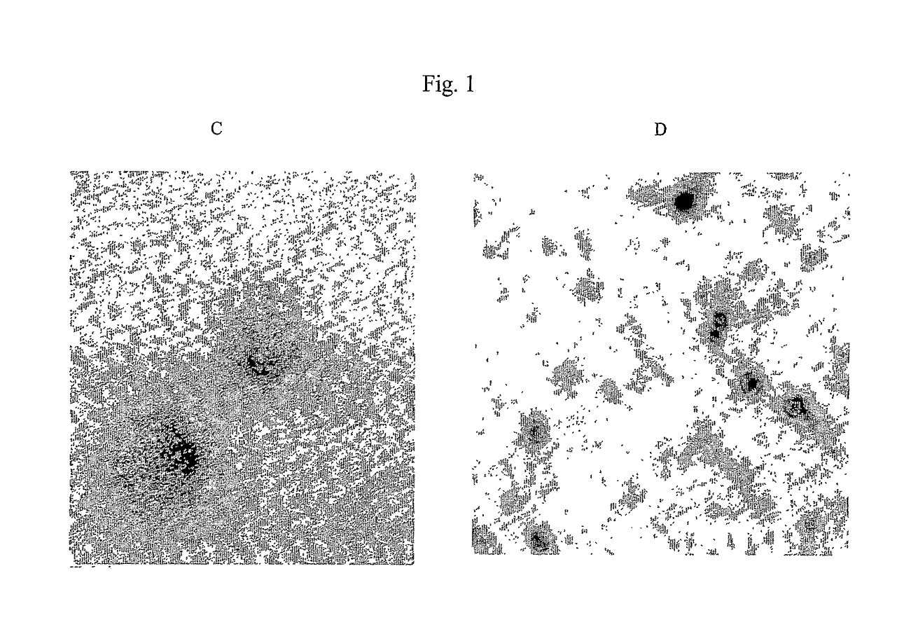

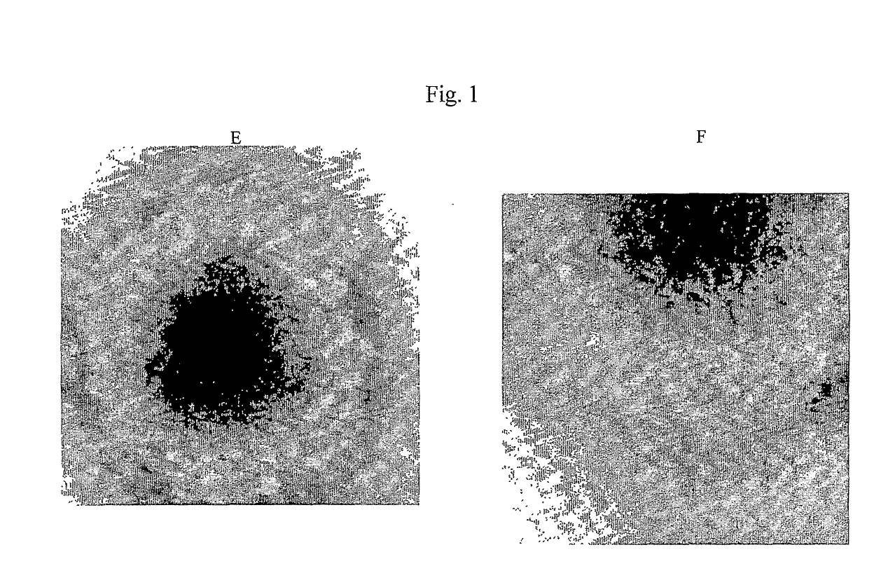

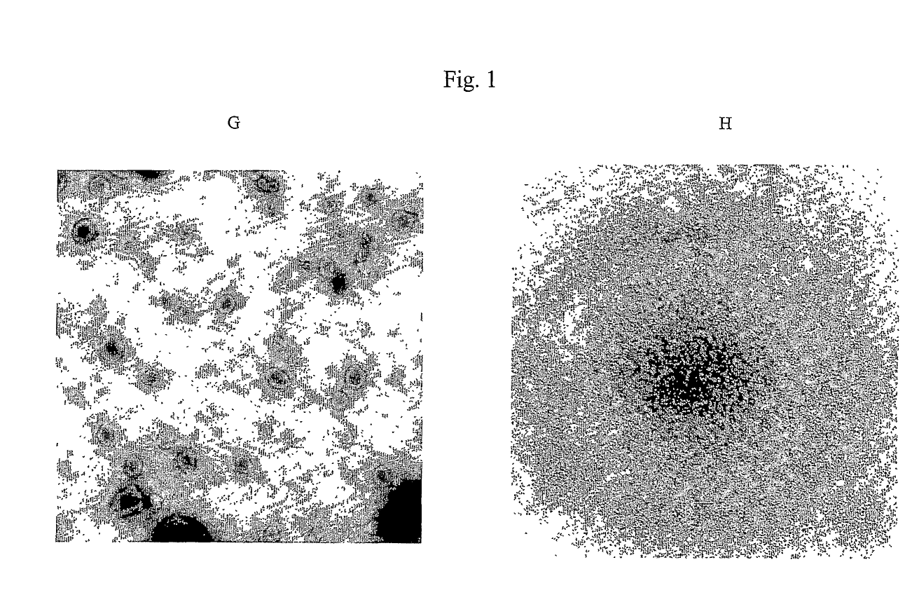

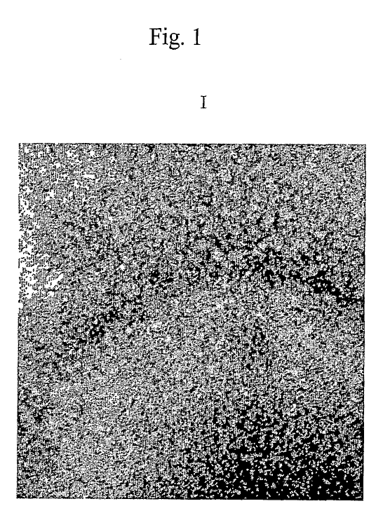

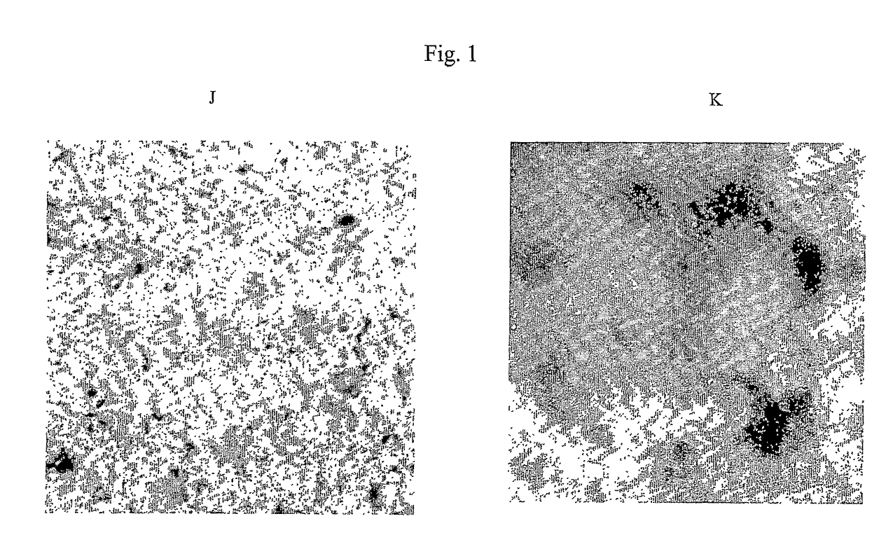

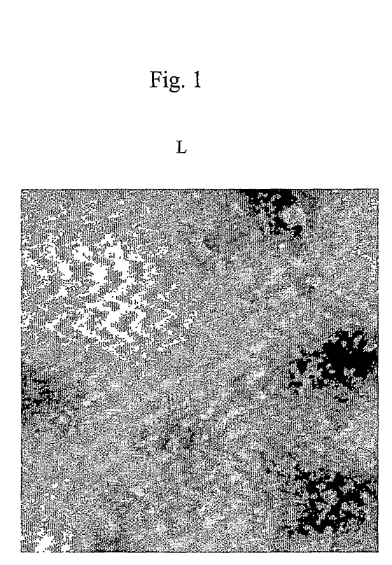

TEM Images of Protein Polypeptides in Aqueous Proteins with 97% water content were obtained. Polypeptides clusters were formed for the samples (3% total protein content) at pH 5.4 and treated with sodium bisulfites. For the control sample, irregular clusters were observed and can be seen in FIG. 1A. The irregular clusters were formed by a mixture of smaller spherical clusters and irregular clusters (see FIG. 1B). As shown in FIG. 1C, the diameter of the spherical cluster was about 50-70 nm. When the ionic strength was increased from 0 to 0.05766, spherical clusters were formed, and the number of such clusters increased as ionic strength increased (See, FIGS. 1D and 1G). The diameter of the spherical clusters also increased with ionic strength. The diameter of the sample treated with 0.02883 ionic strength was from 50-500 nm, while the diameter of the sample treated with 0.05766 ionic strength was from 50-1500 nm. The cluster for the sample treated with 0.02883 ionic strength showed clear boundaries with its environment, as can be seen in FIGS. 1E and 1F, while the sample treated with 0.05766 ionic strength presented a clear line of the sphere, as shown in FIG. 1H. However, numerous polypeptides were tightly attached to the spherical cluster and formed a continuous boundary surrounding the cluster (FIG. 1I). More interestingly, at an ionic strength of 0.1153, these large spherical clusters formed large aggregates (Fig. J), and many small spherical clusters and irregular aggregates were attached to the surface of the large aggregates (FIGS. 1K and 1L).

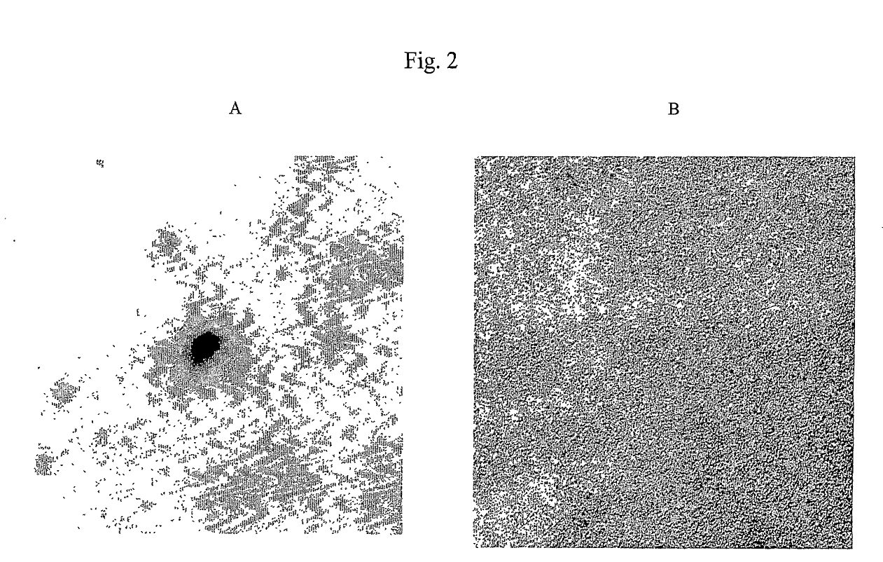

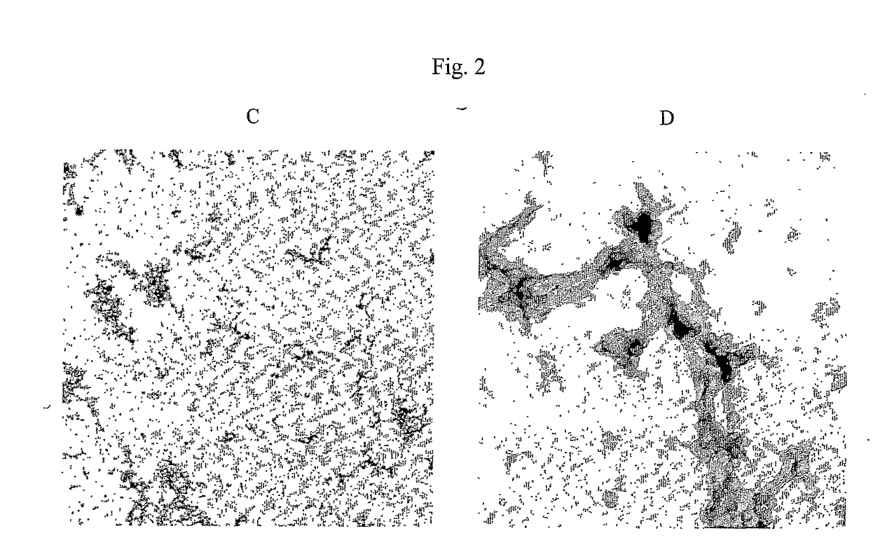

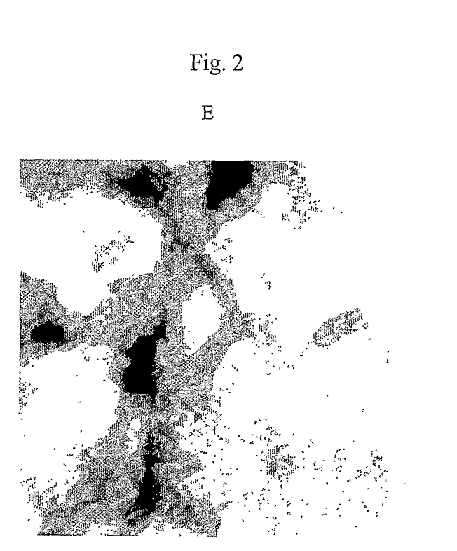

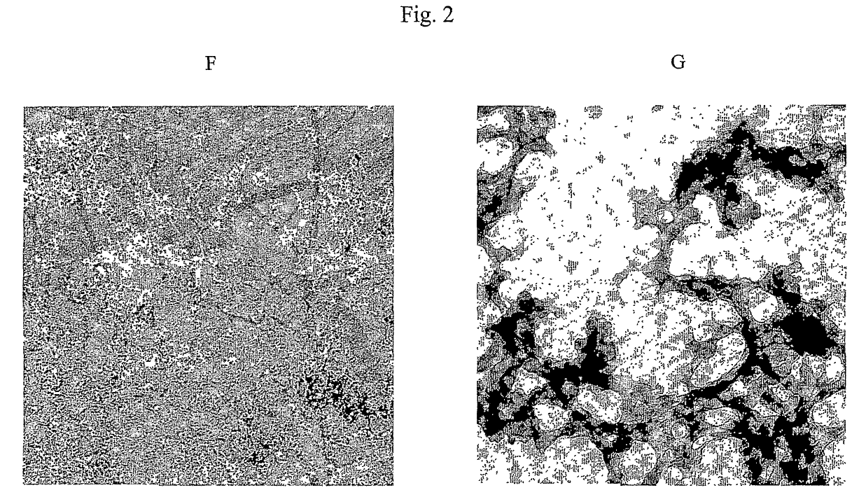

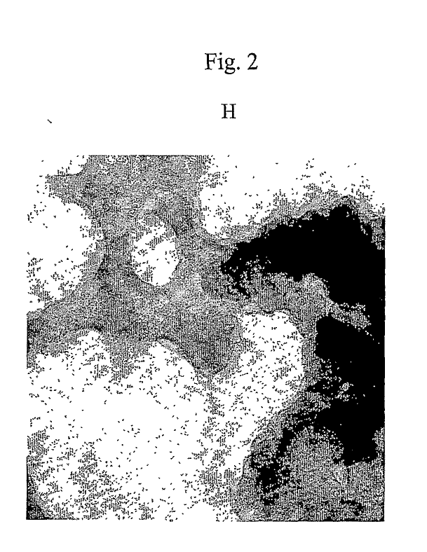

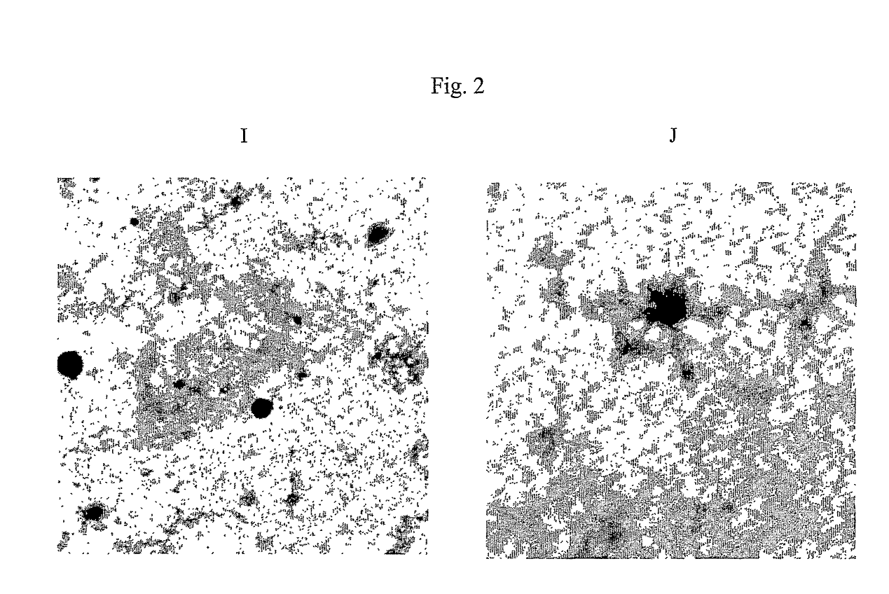

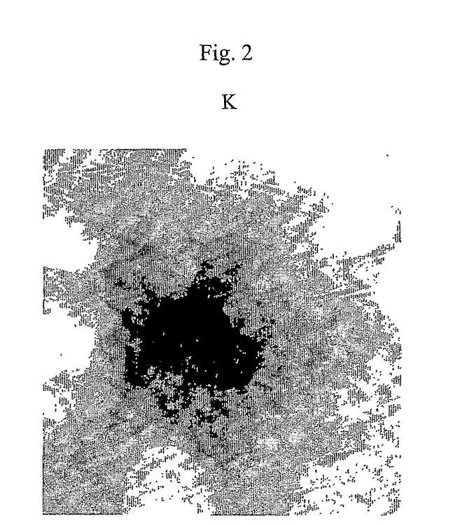

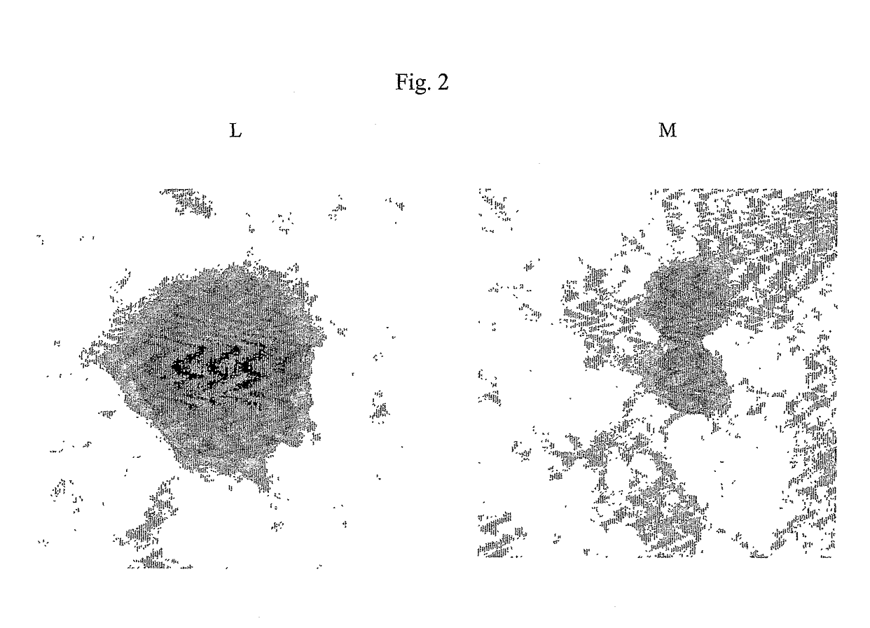

When the pH of the protein solution, with 3% total protein content and treated with sodium bisulfite, was adjusted from 5.4 to 4.8, the spherical clusters turned into a network complex. FIGS. 2D and 2G are typical network complex structures. The degree of such network complex structures increased as the ionic strength increased. The protein samples treated with ionic strengths in the range from 0.02883 to 0.05766 had a uniform complex structure (FIGS. 2. C, D, E, F, G, and H). However, as the ionic strength increased to 0.1153, the degree of the network complex structures increased, and the complex structure was not uniform, as shown in FIG. 2I. Some of the spherical clusters were partially destroyed and became part of the network complex, as shown in FIGS. 2J and 2K, and some even remained as individual or coupled spherical clusters (FIGS. 2L and 2M, respectively). The pH of the control sample was also adjusted from 5.4 to 4.8, no network complex was observed (FIGS. 2A and 2B), but uniform protein precipitation occurred due to the surface charge of the protein being close to neutral because soy protein has an isoelectric point (pI) of 4.5.

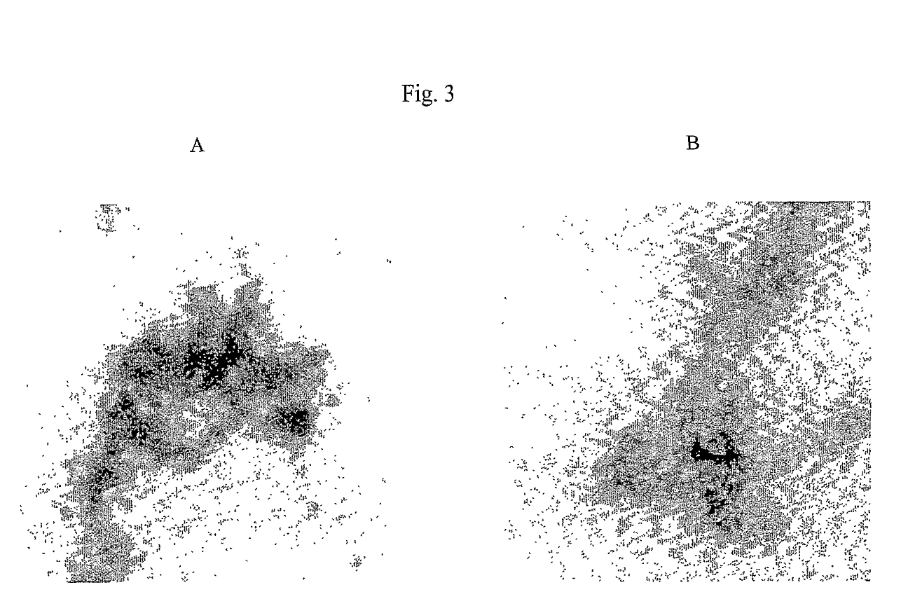

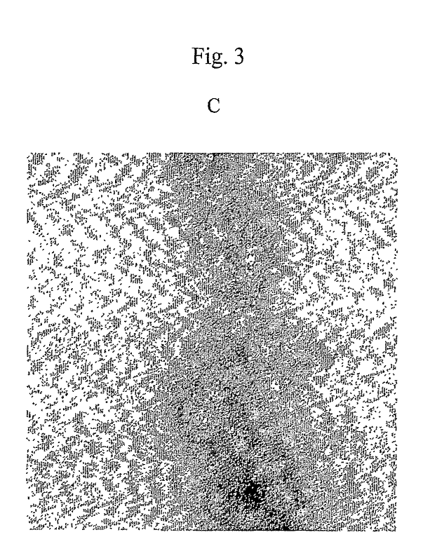

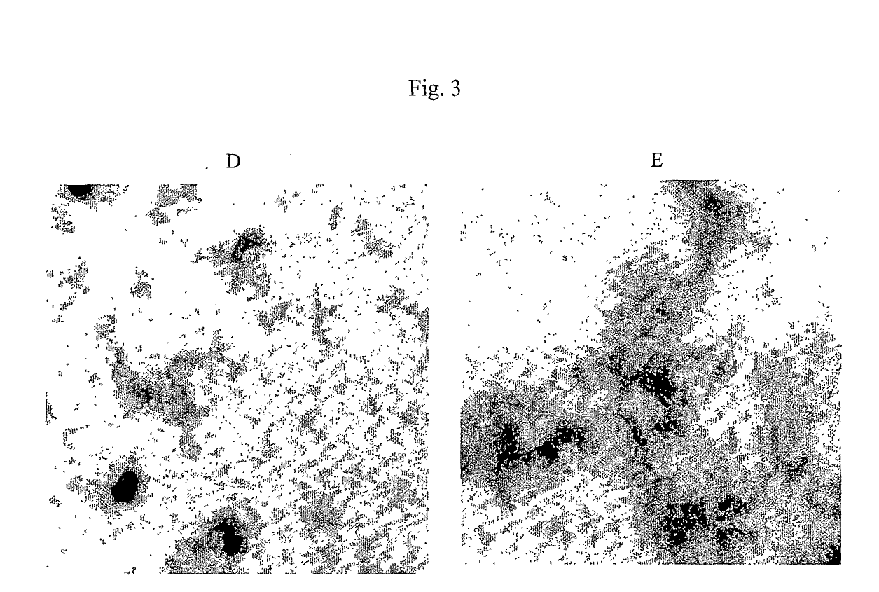

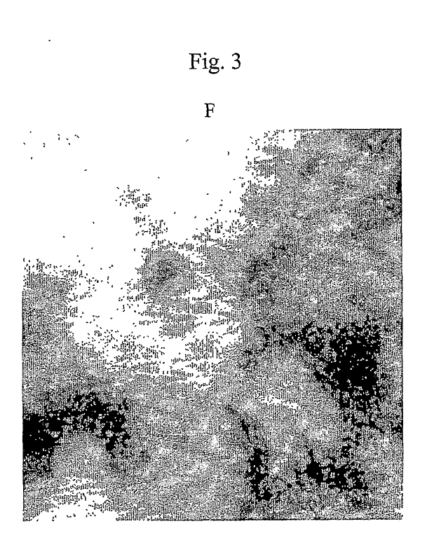

Proteins with 62% water content were then tested. At pH 4.8, when the water content of the protein samples was reduced from 97% to 62%, the network complex shown in FIGS. 2D and 2G became chain-like structures, as shown in FIGS. 3A, 3B, and 3C. The chain was made of numerous small spherical clusters with diameters ranging from 5 to 25 nm. The network complex shown in FIG. 2I also turned into chain like structure, as shown in FIGS. 3D, 3E, and 3F. However, many large spherical clusters remained as tightly aggregated balls with diameters ranging from 100 to 600 nm.

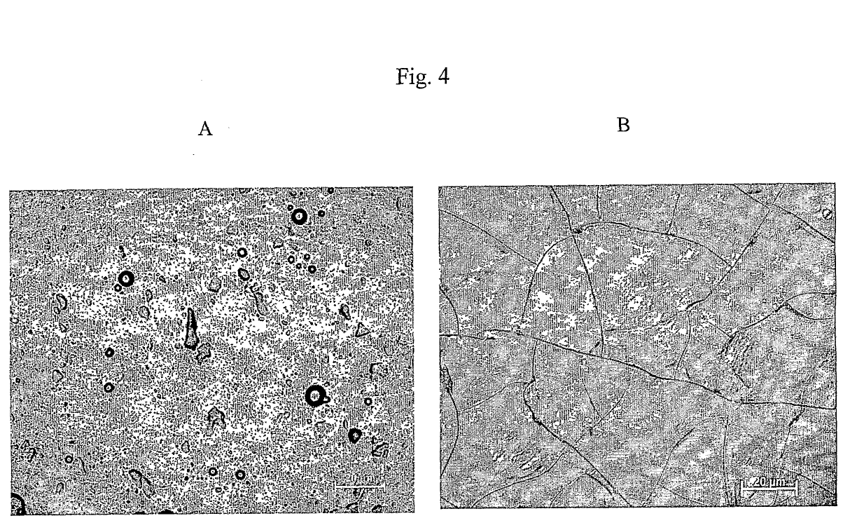

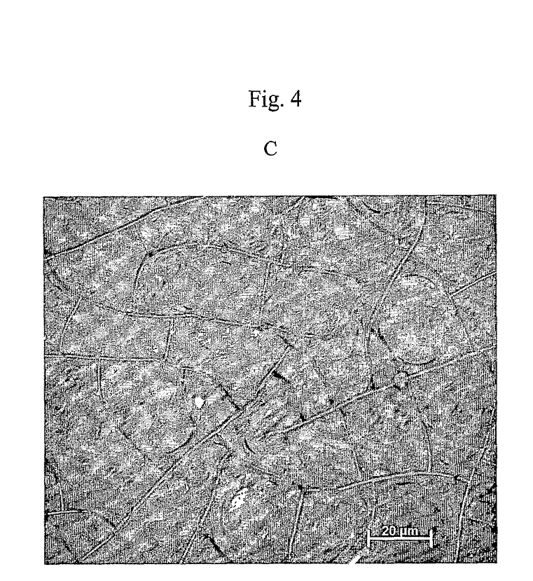

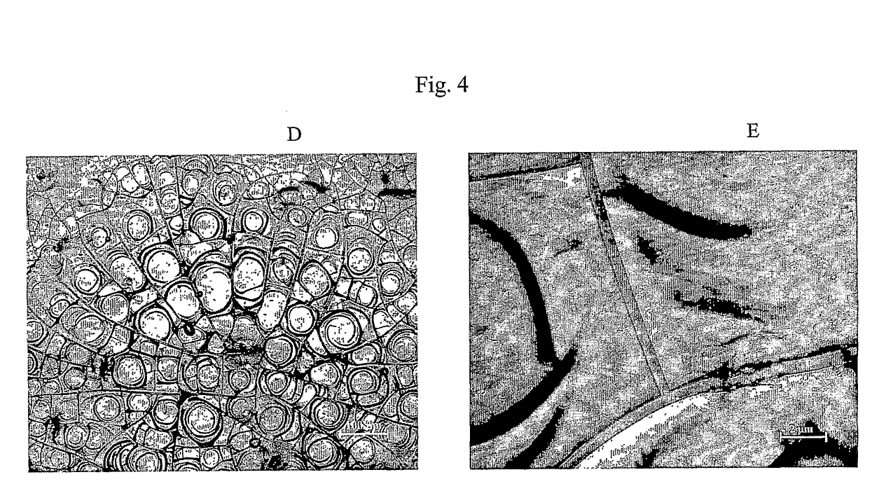

Next, LSM images were taken of the cured proteins. For the protein gels cured at room conditions, the control protein sample without treatment showed a typical uniform brittle structure and protein molecules were individually packed by each other (FIG. 4A). Phase separation was observed for those treated protein samples (FIGS. 4B, 4C, and 4D). Protein samples treated with 0.1153 (12 g sodium bisulfite per liter protein solution) ionic strength formed large circle spider web like structures (FIG. 4D). The component in the phase of the line-like structure shown in FIG. 4E was confirmed to be all hydrophilic amino acids by the SDS-PAGE method and water soaking test. In the circle-like or irregular rectangular shape regions, both hydrophobic and hydrophilic amino acids were found. Most of the hydrophobic parts of the hydrophillic protein were attached to the hydrophobic complex or clusters by hydrophobic bonding.

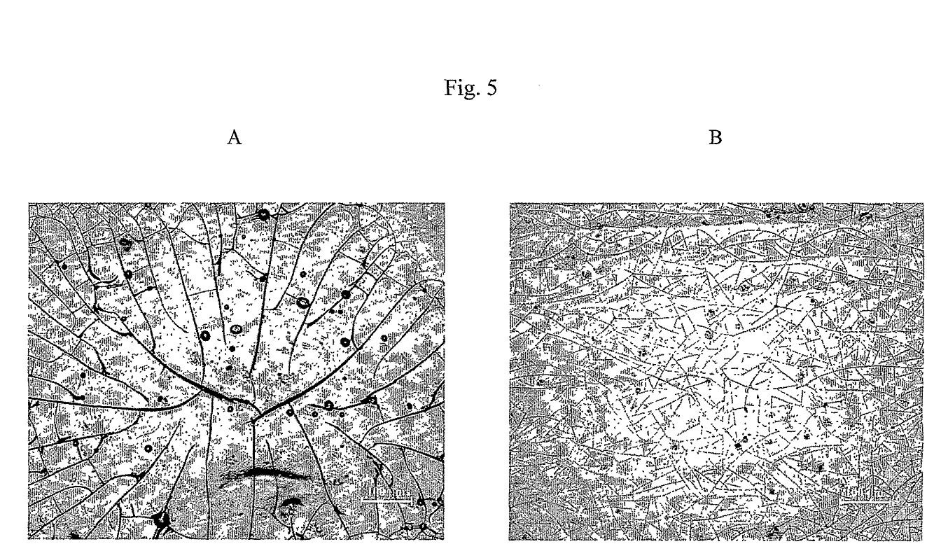

For the water soaking test, the cured protein sample was soaked in distilled water for 48 hours. The line phase migrated into water (FIG. 5). In FIG. 5A, the protein was treated with 12 g/L sodium bisulfite and viewed as a free drop with a small amount of pressure. In FIG. 5B, the protein was treated with 12 g/L sodium bisulfite and spread onto the film with about 1 mm of thickness. The remaining components were collected and analyzed using SDS-PAGE methods that further confirmed the line phase was mainly comprised of hydrophilic proteins.

For the α and β polypeptides, the conglycinin protein was fractioned into α and β polypeptides to further confirm that the clusters, complex, as well as those large spherical like structures were formed by the hydrophobic β polypeptide. X-Ray diffraction results showed that the protein polymers were amorphous and no crystal was observed.

In the rheology analysis, the liquid protein samples exhibited shear thinning flow behaviors as function of the shear rate and temperature, due to those hydrophobic clusters and complex structures.

In thermal analysis testing, the thermal energy used during denaturation of the treated proteins was much less than that used for the native proteins, which further confirmed that the proteins treated with sodium bisulfites became partially unfolded.

### Discussion

Complex formation is due to protein self-association. In this case, hydrophobic bonding is not stronger than self-association. However, some part on the surface of the protein has strong hydrophobic interaction with others, so that complex structures can be formed. When ionic strength is 0.1153, hydrophobic bonding is much stronger, protein self-association is not strong enough to break the cluster. This explains why some of the spherical clusters survived and remained. In this case, the protein liquid was very cohesive and viscous.

By checking the composition of amino acids, the p polypeptide of conglycinin protein contained about 40% hydrophobic amino acids, and the α polypeptide contained about 60% hydrophilic amino acids. The α polypeptide had negatively charged hydrophilic amino acids at one end of its sequence and more hydrophobic amino acids at the other end of its sequence. In contrast, the β polypeptide had a uniform distribution of hydrophobic amino acid segments (3 amino acids in length or longer) throughout its sequence. According to the amino acid sequence of glycinin proteins, basic polypeptide contained more than 40% hydrophobic amino acids, and acidic polypeptide contained more than 60% hydrophilic amino acids. However, based on the TEM and LSM images and rheology properties, basic polypeptide did not show evidence to form spherical clusters or complex structures with the treatments used in this study. However, SDS-PAGE analysis showed that many basic polypeptides were attached to the hydrophobic clusters that may also be caused by hydrophobic bonding. The basic polypeptides contained tryptophan in the middle of their sequences. The tryptophan is likely to form a nuclei for hydrophobic folding that is difficult to unfold. More effective unfolding agents or higher concentrations of unfolding agent can be used and optimized to unfold the basic polypeptides that might also form spherical clusters.

### Example 18

This Example prepares SAIPP and poly(lactic acid) blends. The SAIPP can be prepared in powder form and blended with thermoplastic resins that contain at least one functional group selected from the group consisting of CH₃, OH, COOH, NH₂, SH, per chain length. Those polymers can be either aromatic or aliphatic polymers. The blends can be prepared at the melting temperature of the thermoplastic polymer from room temperature to 230 C. The blends can be used in many was such as a hot glue gun adhesive or extruded into a thin foam noodle or thin foam sheet for other adhesive applications. The blend can be cured by cold press at room temperature. The adhesive can also be used for resin blending with fibers in an extruder for molding composite products. A coupling reagent, such as MDI or MA, with reactive functional groups (i.e., CH3, NH2, NCO, COOH, SH), can be used to improve properties of the blends between SAIPP and other polymers.

### Example 19

SAIPP liquid with about 62% water content was freeze-dried and ground into powder with about 2 mm particle size. In this experiment, polylactic acid (PLA) was used and blended with SAIPP powder at a 30:70 (SAIPP/PLA) ratio at 185C (with or without 0.5% MDI) using an intensive mixture. The SAIPP was easily and uniformly dispersed in the PLA matrix. The PLA's followability was significantly improved. The blend was viscous and sticky. The surface of the blend was smooth and shining with dark brown color. The blend was ground into powder having a particle size of about 2 mm. About 0.2 grams of the powder was used to uniformly cover one piece of a cherry wood sample having a surface area of 20 mm by 125 mm. Another piece of cherry wood sample was used to assemble with the piece with the blend powder and pressed using a hot press at 180 C and 1.4 MPa press pressure for 5 min. The assembled wood specimen was removed from the hot press and set at room temperature for about 10 min. Then the specimen was preconditioned for 2 days in a chamber at 50% relative humidity (RH) and 23 C. The specimen was then cut into five testing samples, each having a 20 mm by 20 mm gluing area. The testing samples were pre-conditioned again in the chamber for another 5 days at 50% RH and 23 C. The dry adhesion strength of SAIPP/PLA (30/70) was in the range of 5.5 to 6.6 MPa with 100% wood failure. The dry adhesion strength of PLA with cherry wood substrate was about 3.5 MPa with adhesion failure.

The resin derived from SAIPP and PLA should have great potential for engineered cellulosic (i.e., wood) based composites that are 100% bio-based. Most currently used resins for engineered wood products are made from petroleum based plastics.

### Example 20

Similar experiments as those conducted in Example 19. In this example, the ratio of SAIPP and PLA was 10/90 and there was no MDI coupling reagent. Procedures used for blending the SAIPP and PLA are the same as in Example 19 except that the blending temperature was 175 C. Procedures used for wood specimen preparation were the same as those used in Example 18 except for that no pre-conditioning was used. Adhesion dry strength was 4.8 MPa with fine wood fiber failure. Adhesion strength should be higher after conditioning.

### Example 21

The SAIPP was blended with poly vinyl acetate (PVAc) using an intensive mixture at a ratio of 10/90 (SAIPP/PVAc) at 140 C. Cherry wood was used for adhesion testing specimen preparation. Procedures used for adhesion testing were the same as those used in Example 18 except for that no pre-conditioning was used. Dry adhesion strength was 3.5 MPa with adhesive cohesive failure. PVAc is a key component of current latex adhesives. Blending SAIPP with PVAc will reduce the usage of PVAc.

### Example 22

The SAIPP/PVAc blends prepared in Example 21 were dissolved in ethanol and no SAIPP particles were observed in the solution, showing that SAIPP and PVAc are compatible.

In addition to the soy latex-like adhesives described above, the SAIPP can be used in an aqueous form that can be blended with either hydrophobic or hydrophilic polymers or resins in liquid form (these polymers or resins can be either in aqueous or nonaqueous) for a variety of adhesive and/or paint applications.

In this example, SAIPP in liquid form was blended with Elmer's glue at a ratio of 70/30 (SAIPP/Elmer's glue). The blend was uniform and there was no phase separation. Adhesion performance of this blend was not evaluated. However, for children's art works, the SAIPP can be used alone or blended with the Elmer's glue to reduce the usage of synthesized latex formula and PVAc.

### Example 23

This Example blended liquid form SAIPP with epoxidized plant oil, such as epoxidized soybean oil (ESO). The ring of the ESO can be opened by using a catalyst, such as boron trifluoride dimethyl etherate (BF₃), or lithium stearate. The NH₂ groups from SAIPP act as a curing agent of the ESO. Coupling reagent or curing agent for ESO can also be added. The SAIPP can also be blended with ESO directly or ratios of ESO with ring-opening ESO for self-healing paint materials, can be made.

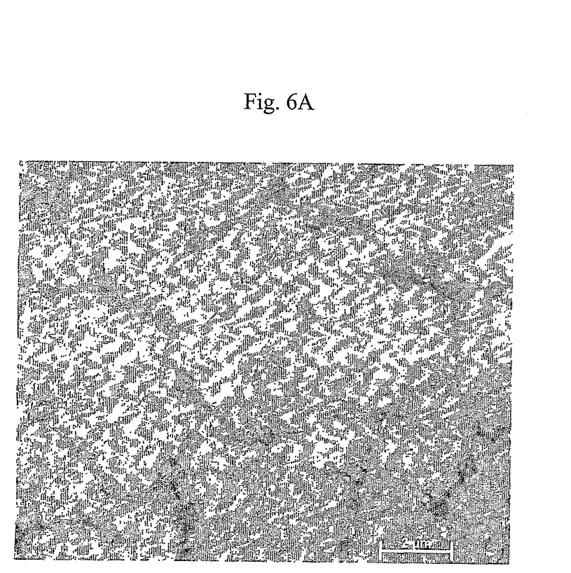

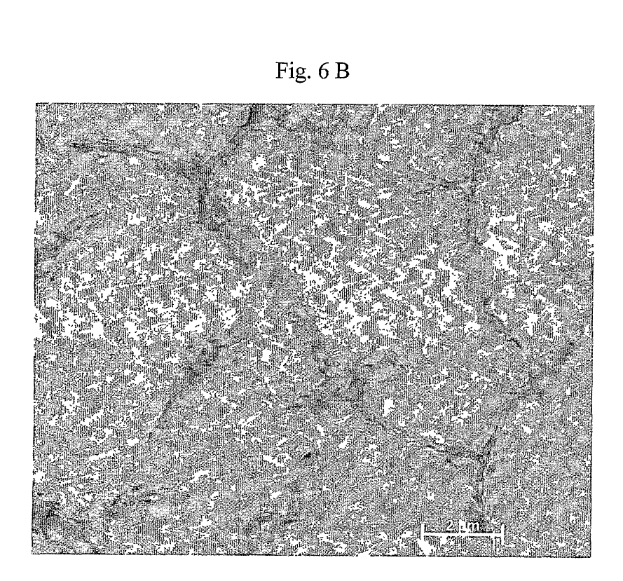

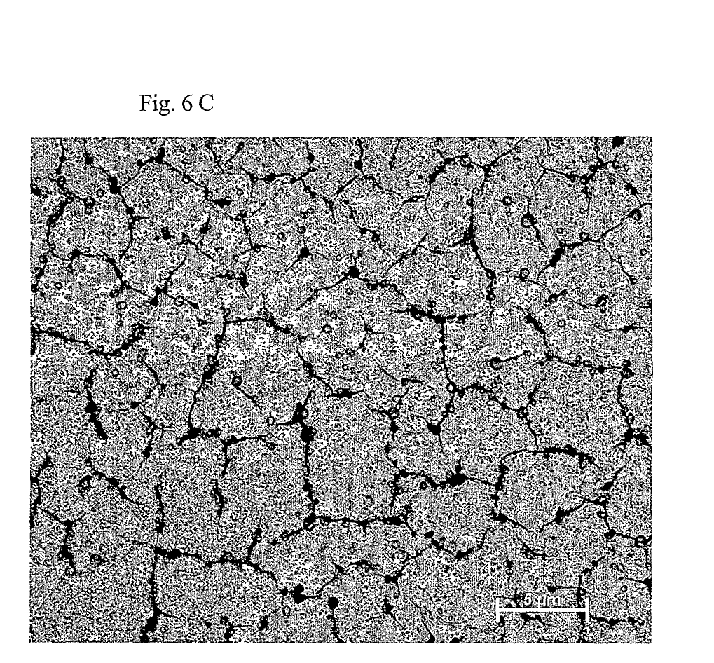

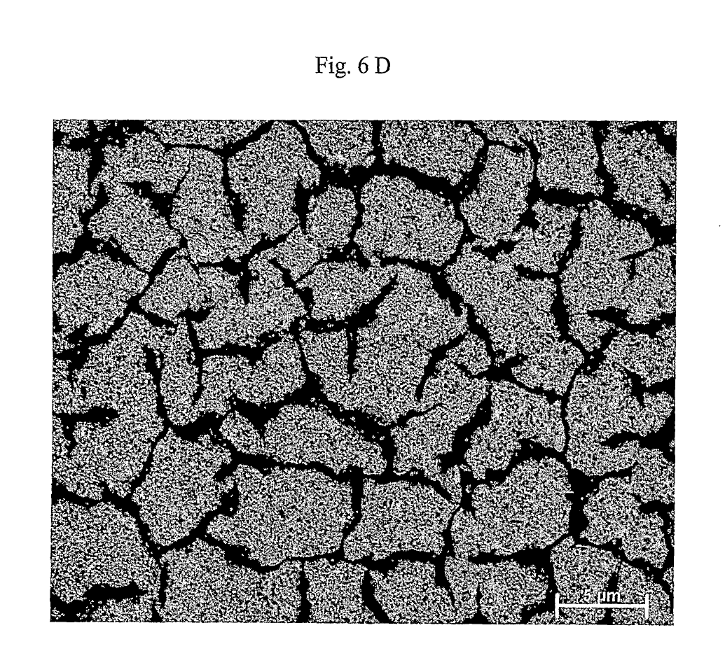

Due to hydrophobic aggregation during curing, when the SAIPP was coated onto a piece of paper, the paper became bent due to the internal stress. In this case, the adhesive strength between the SAIPP and paper was stronger than the hydrophobic aggregation. There was no phase separation, but the trade off was the paper bending. When the SAIPP was coated onto a wall or glass or wood, phase separation was observed at the location where the film thickness was more than 0.5 mm. 3% of ESO was blend with SAIPP with 62% water content. The ESO was uniformly distributed in the SAIPP due to hydrophobic bonding. Upon curing, some of the ESO molecules became debonded with SAIPP as needed to heal any gaps due to phase separation. FIGS. 6A and 6C show the two images before and after water soaking. These images show that the hydrophilic components were covered by the ESO and no substance was found in the water. Additionally, the coating surface remained the same, even after being soaked in water. FIGS. 6B and 6D show that the SAIPP with 3% ESO in large scale before and after water soaking, the phase separation caused by hydrophobic aggregation was uniform and controlled. In addition, without ESO, the hydrophilic components (the lines showed in FIG. 4) dissolved into water and caused cracks. The ESO healed the phase separation and prevented cracking. It is good for coating and paint applications.

The paper coated with the SAIPP with 3% ESO exhibited a smooth surface and no bending phenomena occurred. The stress caused by the hydrophobic aggregation was released by the ESO debonding.

### Example 24

This Example prepares a soy flour-based SAIPP blended with Phenol formaldehyde (PF) resin. The soy flour had a protein content of approximately 50%. The soy flour was dissolved in water to a 12% solids content. The pH of the soy flour solution was adjusted to 9.5, and 6 g/L NaHSO₃ was added to the solution before being stirred for two hours. The pH of the soy flour solution was then adjusted to 7.0. Next, PF resin (Georgia Pacific Resin, Inc) was added to the solution at a ratio of 1:1 (PF:soy flour) and stirred for a few minutes at room temperature. Cherry wood was used as the substrate for plywood testing. The blended resin was uniformly applied to each piece of the cherry wood sample with a surface area of 20 mm by 125 mm. Two pieces of wood samples were used in this experiment. The wood samples were assembled after 10 minutes of the resin brushing, and pressed using a hot press at 170 C and 1.4 MPa press pressure for 5 minutes. Next, the specimen was preconditioned for 2 days in a chamber with 50% RH and maintained at 23 C. The specimen was then cut into five testing samples with 20 mm by 20 mm gluing area. The testing samples were pre-conditioned again in the chamber for another 5 days at 50% RH and at 23 C. The dry adhesion strength of SAIPP/PF was in the range of 5.5 to 6.0 MPa with 100% bulk wood failure. Wet adhesion strength was tested by soaking the specimen in tap water for 48 hours under typical room conditions and tested while still wet. Wet adhesion strength was 3.7 MPa with 80% wood fiber failure. These are similar to PF alone.

### Example 25

This Example prepares and tests a SAIPP made with NaCl. To begin, 0.5 mol of NaCl was dissolved in distilled water. Soy protein isolate was dissolved in the NaCl solution at a 28% solids content. The pH of the slurry was adjusted to 9.5 and stirred for two hours. Then the pH was adjusted to 4.8 as the SAIPP solution. The SAIPP was then applied for adhesion testing. Procedures of adhesion testing are the same as those used in Example 24. Dry adhesion strength was 5.8 MPa with 100% bulk wood failure, wet strength was 3.5 MPa with 65% wood failure, and boiling wet strength was 2.8 MPa with 40% wood fiber failure.

We claim:

1. An adhesive composition comprising a protein and a protein unfolding agent, wherein the pH of said composition is near the isoelectric point or percolation level of said protein.

2. The composition of claim 1, said protein being a vegetable protein.

3. The composition of claim 1, said protein unfolding agent being selected from the group consisting of ionic compounds, NaHSO₃, NaCl, p-hydroxybenzoic acid ethyl ester, p-hydroxybenzoic acid methyl ester, Na₂S, dehydroacetic acid, n-propyl p-hydroxybenzoate, sodium sulfite, 2-mercaptoethanol, and combinations thereof.

4. The composition of claim 1, further comprising an additive selected from the group consisting of cellulose, resins, polymers, coupling agents, a second protein source, oil, and combinations thereof.

5. A method of forming a protein-based polymer comprising the steps of:
forming a solution comprising water, a first protein, and a protein unfolding agent; and
adjusting the pH of said solution to near the isoelectric point or percolation level.

6. The method of claim 5, said protein unfolding agent being selected from the group consisting of ionic compounds, salts, detergents, urea, reducing agents, and combinations thereof.

7. The method of claim 5, said protein unfolding agent being selected from the group consisting of NaHSO₃, NaCl, p-hydroxybenzoic acid ethyl ester, p-hydroxybenzoic acid methyl ester, Na₂S, dehydroacetic acid, n-propyl p-hydroxybenzoate, 2-mercaptoethanol, sodium sulfite, and combinations thereof.

8. The method of claim 5, said first protein being derived from plant or animal sources.

9. The method of claim 5, further comprising the step of reducing the water content of said solution.

10. The method of claim 5, further comprising the step of adding an additive selected from the group consisting of cellulose, resins, polymers, coupling agents, a second protein source, oil, and combinations thereof.

11. The method of claim 10, said additive being selected from the group consisting of epoxidized plant oil, polylactic acid, thermoplastic polymers, thermoplastic resins, polyvinyl acetate, children's glue, and combinations thereof.

12. The method of claim 5, further comprising: the step of centrifuging said solution.

13. The method of claim 5, further comprising the step of refrigerating said solution.

14. The method of claim 5, further comprising the step of adjusting the ionic strength of said solution.

15. The method of claim 5, further comprising the step of adjusting the pH of said solution a second time.

16. A method of preparing an adhesive comprising the steps of:
combining a protein source, water, and a protein unfolding agent to produce a solution;
adjusting the pH of said solution to above about 6;
centrifuging said solution to remove carbohydrates and provide a first supernatant;
adjusting the pH of said first supernatant to an acidic level;
refrigerating said pH-adjusted supernatant;
centrifuging said refrigerated supernatant to produce a first precipitate and second supernatant; and
using said second supernatant or said first precipitate as said adhesive.

17. The method of claim 16, further comprising the steps of:
adjusting the pH of said second supernatant to an acidic level to form a second solution;
centrifuging said second solution to produce a third supernatant and second precipitate;
adding water to said third supernatant;
adjusting the pH of said third supernatant to be near the isoelectric point or percolation level of said protein;
refrigerating said pH-adjusted third supernatant;
centrifuging said refrigerated, pH-adjusted third supernatant to produce a fourth supernatant and third precipitate; and
using said third precipitate as an adhesive.

18. The method of claim 16, said protein unfolding agent being an ionic compound selected from the group consisting of salts, detergents, urea, reducing agents, and combinations thereof.

19. The method of claim 16, said protein unfolding agent being selected from the group consisting of NaHSO₃, NaCl, p-hydroxybenzoic acid ethyl ester, p-hydroxybenzoic acid methyl ester, Na₂S, dehydroacetic acid, n-propyl p-hydroxybenzoate, 2-mercaptoethanol, sodium sulfite, and combinations thereof.

20. The method of claim 16, said protein source being derived from plant or animal sources.

21. A method of producing an adhesive comprising the steps of:
combining a protein source and water to produce a slurry;
adjusting the pH of said slurry to a basic level;
adding a protein unfolding agent to said pH-adjusted slurry to form a second slurry;
adjusting the pH of said second slurry to an acidic level;
centrifuging said pH-adjusted second slurry to produce a first supernatant;
adjusting the pH of said first supernatant to an acidic level;
centrifuging said pH-adjusted first supernatant to produce a first precipitate and a second supernatant; and
using said first precipitate as an adhesive.

22. The method of claim 21, further comprising the step of centrifuging said second slurry to remove carbohydrates and carbohydrate-related material.

23. The method of claim 21, further comprising the steps of:
centrifuging said second supernatant to produce a second precipitate; and
mixing said first and second precipitates to produce an adhesive.

24. The method of claim 21, further comprising the step of:
curing said adhesive.

25. The method of claim 21, said protein unfolding agent being an ionic compound selected from the group consisting of salts, detergents, urea, reducing agents, and combinations thereof.

26. The method of claim 21, said protein unfolding agent being selected from the group consisting of NaHSO₃, NaCl, p-hydroxybenzoic acid ethyl ester, p-hydroxybenzoic acid methyl ester, Na₂S, dehydroacetic acid, n-propyl p-hydroxybenzoate, 2-mercaptoethanol, sodium sulfite, and combinations thereof.

27. The method of claim 21, said protein being derived from plant or animal sources.

28. A method of producing an adhesive comprising the steps of:
combining protein source and water to produce a slurry;
adjusting the pH of said slurry to a basic level;
centrifuging said pH-adjusted slurry to produce a first supernatant without carbohydrates;
adjusting the pH of said first supernatant to an acidic level;
centrifuging said pH-adjusted first supernatant to produce a second supernatant;
adjusting the pH of said pH-adjusted second supernatant to produce a precipitate;
adding an unfolding agent to said precipitate; and
stirring said precipitate and said folding agent to produce said adhesive.

29. The method of claim 28, said protein unfolding agent comprising NaHSO₃.

30. The method of claim 28, said protein source being derived from plant or animal sources.

31. A method of producing an adhesive comprising the steps of:
combining a protein source and water to produce a slurry;
adjusting the ph of said slurry to a basic level;
adding an unfolding agent to said slurry;
adjusting the pH of said slurry to an acidic level; and
adjusting the pH of said pH-adjusted slurry to a desired level to produce an adhesive.

32. The method of claim 31, said protein unfolding agent comprising NaHSO₃.

33. The method of claim 31, said protein source being derived from plant or animal sources.
